(19) United States
(12) Patent Application Publication
(10) Pub. No.: US 2011/0166027 A1
(43) Pub. Date: Jul. 7, 2011

(54) INTERACTION SCREENING METHODS, SYSTEMS AND DEVICES

(75) Inventor: Michael P. Weiner, Guilford, CT (US)

(73) Assignee: AFFOMIX CORPORATION, Branford, CT (US)

(21) Appl. No.: 12/674,374

(22) PCT Filed: Aug. 21, 2008
(86) PCT No.: PCT/US08/73879

§ 371 Date: Jun. 22, 2010

Related U.S. Application Data
(60) Provisional application No. 60/956,966, filed on Aug. 21, 2007.

(51) Int. Cl. C40B 20/04; C40B 30/04; C12N 1/00

(52) U.S. Cl. 506/4; 506/9; 435/243

(57) ABSTRACT

An interaction screening method for identifying binding moieties encapsulates candidate binding moieties in droplets with a first known moiety and a second known moiety. The candidate binding moieties are different in the different droplets. The method further comprises determining for one or more of the droplets whether the candidate binding moiety is bound to the first known moiety and/or the second known moiety. Optionally, the method further comprises segregating at least one droplet in which the candidate binding moiety is bound to the first known moiety or to the first and second known moiety.

### PRIORITY CLAIM

This application claims priority to U.S. Provisional Application Ser. No. 60/956,966, filed Aug. 21, 2007 and entitled, Microfluidic Interaction Screening, the entire disclosure of which is hereby incorporated by reference.

### INTRODUCTION

The present invention is directed to methods of performing interaction screening and, in particular, to microfluidic methods and systems for performing interaction screening to identify one or more antibodies or other moieties that interact selectively with a target entity.

### BACKGROUND

Various techniques have been developed to identify entities that interact, e.g., to identify antigen-antibody, protein-protein, protein-nucleic acid, protein-ligand, nucleic acid-nucleic acid, cell-protein, cell-cell, ligand-ligand and ligand-receptor interactions. In the case of antibodies (“Abs”), e.g., generation of Abs against an antigen has typically involved a costly and time-consuming processes of obtaining and purifying the target antigen (“Ag”), e.g., a protein or other Ag, injecting the Ag into laboratory animals (often requiring multiple injections over several weeks), and attempting to select polyclonal Abs or Ab-producing cells in the case of monoclonal Abs (“mAbs”), such that the selected Abs (or mAbs) possess the requisite affinity and selectivity for the Ag. Using current interaction screening techniques for phage display, an in vitro alternative for the identification and isolation of Abs for a target antigen, considerable amounts of purified Ag are required, particularly for the characterization of affinity and specificity of the Abs selected in the screens.

Likewise, yeast two-hybrid (Y2H) interaction screening provides an approach to generation and selection of mAbs in which intracellular expression of the Ag eliminates the need for Ag production and purification. However, in the Y2H approach as it has previously been implemented, the bait is typically a protein and mAbs cannot readily be selected against non-protein Ags. In addition, proteins are not post-translationally modified in yeast as they are in higher mammals, so that identifying Abs in Y2H that differentiate among states of post-translational modification (PTM) presents particular challenges using previously known techniques.

Known techniques for hybridoma and phage display, illustrated in FIGS. 1A and 1B, respectively, typically require milligram amounts of purified proteins for screening and activity assays. These techniques often involve inefficient screening (false positives, non-productive hybridomas, low plating efficiencies, etc.), and there is a need to validate that the selected Abs possess desired degrees of specificity for their cognate Ags. Protein production and/or purification can be rate limiting: it is expensive, time-consuming and often difficult. The screening inefficiencies and need to validate specificity result in increased costs and labor, as well as reduced throughput.

In FIG. 1 mAb production by mouse hybridoma cells is seen to start with immunization of animals with a selected Ag to stimulate Ab-forming immune cells to produce a range of Abs with varying specificities and potencies. Collected immune cells are fused with tumor (myeloma) cells to produce immortalized hybridoma cells, each producing a mAb with a distinctive reactivity. These hybridoma cells are then screened in vitro for those with reactivities against the Ag of interest, and specific clones are isolated, e.g., by limiting dilution. The selected cells are grown by clonal expansion, and a single population of mAbs is harvested from each clone. FIG. 1B shows production of mAbs by an exemplary phage display technique, specifically, M13 Bacteriophage Biopanning. Sequential panning and infection cycles are carried out to enrich for phage that bind to a “bait”, which is attached to a solid support. The phagemids are rescued in E. coli and individual selections can be assayed by superinfection with M13 helper phage to produce phage for a 96-well enzyme-linked immunosorbent assay” (“ELISA”).

A microfluidics-based hybridoma system has been developed by Dr. David Weitz and colleagues at Harvard University, as illustrated in FIG. 7, for rapid screening of many cells that are potential mAb producers. Hybridoma cells secreting mouse mAbs are seen in FIG. 7 to be incubated in droplets with a bead coated with a target Ag. The droplets are merged with a droplet containing a fluorescently-labeled secondary Ab directed against mouse Abs, e.g., fluorescein-labeled secondary Ab. The droplet is then passed through a narrowing in the flow channel such that the bead(s) flow past a detector in single file. Read-out occurs on a bead-by-bead basis. The location of the secondary Ab is measured and droplets having fluorescently-labeled beads are sorted. In such design developed by Weitz et al., hybridoma cells secreting mouse mAb are incubated in droplets with beads coated with an Ag. If the hybridoma produces a mAb that interacts with Ag attached to a bead, the secondary Ab will bind to the mAb, creating an area of fluorescence at the bead site. Droplets are then sorted such that droplets containing fluorescein-labeled beads are separated from those that do not. The hybridoma cells can survive in droplets and be recovered, and it is estimated that one cell requires about 50 pL of culture medium per hour in order to survive. In this regard, it has been observed that survival time is correlated with drop size. Incubation time for a typical screen is about 4-5 hours. By adjusting medium content and droplet size, one is able to achieve greater than 75% cell survival and recovery within droplets for at least 7 hours. FIG. 8 shows cell survival and medium content screening in microfluidics. Channel pinch and nuclei separation resolve multiple cells and beads in single droplets. The graph figure on the right indicates percentage of live vs dead cells when measured after 7 hours.

Conventional production of mAbs against a specific Ag is time consuming, typically requiring 3-6 months for selection of a desired mAb against a particular target, and costly, typically costing approximately $8000 per mAb. In addition, mAbs are selected based on affinity for Ag target but are not necessarily differentiated or selective for reactivity against the Ag target. It is well established in the art that many selected Abs are found not to be useful because of undesirable cross reactivities. Finally, there are limitations on the use of relatively fragile hybridoma cells.

It is an object of at least certain aspects of the invention disclosed here to provide methods of performing interaction screening, e.g., to identify one or more candidate binding moieties that interact with a target moiety, and optionally to isolate such identified candidate binding moieties from other moieties that do not bind to the target. It is an object of at least certain aspects of the invention disclosed here to provide methods of obtaining candidate Abs or other moieties that bind selectively with an Ag or other target moiety, by interaction screening to identify candidates that do bind selectively to the target and optionally to isolate them from others that do not. It is an object of at least certain aspects of the invention disclosed here to provide systems operative to perform such methods, i.e., capable of performing such interaction screening and, in at least certain embodiments also to select the candidate Abs or other entities, e.g., a library of such candidates, and to sort or isolate one or more reactive or specifically reactive candidates from those that are not. It is an object of at least certain methods of the invention to perform interaction screening by certain novel microfluidics techniques. It is an object of at least certain embodiments of a system aspect of the invention to provide systems operative to perform interaction screening by such microfluidics techniques. It is an object of at least certain exemplary system and method embodiments of the disclosed invention to identify Abs, e.g., mAbs that bind selectively to a target Ag, and to isolate such Abs from other candidates that do not bind selectively to the target Ag. Certain such method and system embodiments of the invention identify Abs that bind to a target Ag, discriminating them from other Abs which have non-specific or non-desirable binding properties, e.g., binding affinity to non-target moieties. Additional objects and features of the invention will be apparent to those of ordinary skill in the art given the benefit of this disclosure.

### SUMMARY

In accordance with one aspect, an interaction screening method for identifying one or more candidate binding moieties based on binding to one or more known other moieties, comprises providing droplets of liquid medium, at least a majority of which contain at least one candidate binding moiety. The candidate binding moiety may be encapsulated in the droplets directly as the moieties themselves or may be provided by encapsulating microbes which then secrete or otherwise provide the candidates m in the droplets. The droplets further have a first known moiety and a second known moiety. The candidate binding moiety is different in the different droplets. The phrase “different in the different droplets” and all similar phrases used here and elsewhere in this disclosure with reference to this or other aspects of the invention, means that the candidate binding moiety in at least most (i.e., more than half, e.g., almost all) or all of the droplets is different from the candidate binding moiety in most or all others of the droplets. The interaction screening method further comprises determining for one or more of the droplets whether the candidate binding moiety is bound to the first known moiety and whether the candidate binding moiety is bound to the second known moiety. In certain exemplary embodiments such determinations are made for at least most of the droplets. Certain exemplary embodiments of the disclosed interaction screening method, after the determination step, further comprise segregating from others of the droplets at least one droplet in which the candidate binding moiety of that droplet is bound to the first known moiety. In certain other exemplary embodiments, the method comprises, after the determination step, segregating from others of the droplets at least one droplet in which the candidate binding moiety of that droplet is bound to the first known moiety and to the additional known moiety.

Those of ordinary skill in the art will understand from this disclosure, that in different applications and different embodiments of the methods, systems and devices disclosed here, determination of whether or not binding occurs between the candidate binding moiety(ies) contained in a given droplet with the first known moiety, the second known moiety and any additional known moieties included in the droplet can provide information about the properties of the candidate as a binding moiety. A successful test may involve binding to one and not the other, or in other cases binding to both (or all) of the known moieties in the droplet. For example, differential binding may provide information relevant to binding specificity, or to evaluate binding to proteins comprising part or all members encoded by a gene family.

Certain exemplary embodiments of the disclosed interaction screening method segregate from others of the droplets at least one droplet in which the candidate binding moiety of that droplet is bound to the first known moiety and not bound to an additional known moiety. In certain exemplary embodiments some or all of the droplets of liquid medium further contain one or more additional known moieties, and the method further comprises determining for one or more of the droplets whether the candidate binding moiety is bound to the one or more additional known moieties. As used here, the first known moiety, the second known moiety and any additional known moieties are known in the sense that their molecular or macromolecular structure is at least partly known or that the moiety has been otherwise identified for interaction screening against the candidate binding moieties.

In at least certain exemplary embodiments the first known moiety may be a target Ag and the candidate binding moieties may be possible Abs for such target Ag. The second known moiety in the droplets in certain embodiments is a non-target moiety in that, e.g., binding of a given droplet's candidate binding moiety to the second known moiety would indicate lack of specific binding of that droplet's candidate binding moiety to the first known moiety. In yet other embodiments the determination of binding of the candidate binding moiety to the second known moiety would serve an additional or different purpose. The optional inclusion of one or more additional known moieties for interaction screening likewise can serve similar such purposes.

It should be understood, that in many embodiments of the methods, systems and devices in accordance with the first aspect of the invention disclosed above or other aspects disclosed below, any given droplet is different from most but necessarily all of the other droplets of the collection. For example, more than one of the droplets may contain the same candidate binding moiety. But at least most of the droplets have a candidate binding moiety that is different from the candidate binding moiety of at least most others of the droplets. For example, in embodiments wherein candidate binding moieties are provided in the droplets by encapsulating recombinant microbes which then secrete or otherwise provide the candidate binding moieties in the droplets, each genetic variant of the microbe may occur in a small number of the droplets. Thus, the statement, “the microbe of one droplet would be genetically different from the microbes of other droplets” (and all similar such descriptions herein), means that the microbe in any given droplet is different from the microbe in many or most of the other droplets, but is not necessarily different from the microbe in all of the other droplets. For example, in order to distribute a library of 10⁸ genetic variants of a microbe into 10⁹ droplets for a given embodiment of the methods, systems or devices disclosed here, there would be, on average in the resulting collection of droplets, about 10 droplets that have each of the microbe variants. Typically, a Poisson distribution would be expected rather than precisely 10 droplets each, and some droplets may have multiple microbes and may for that reason be more or less useful. Certain exemplary embodiments wherein some of the droplets or even many of the droplets have multiple microbes (or multiple candidate binding moieties directly encapsulated into the droplets) can be in some cases an efficient screening strategy using the methods, systems and devices disclosed here. In any event, in such embodiments each droplet is different from other droplets (in the sense that concept is used here) in that each droplet is different from the vast majority of other droplets, notwithstanding that any given droplet may be the same as, e.g., none, 1, 5, 10, 20 or even a hundred or more other droplets in the collection. Similarly, as another example, if a library of candidate binding moieties used in a method, system or device according to this disclosure has 10⁸ different members, and the library is encapsulated in 10⁹ droplets, each candidate will, on average, be in about ten droplets. In such embodiments, each droplet is different from other droplets of the collection in the sense that each is different from the vast majority of other droplets, notwithstanding that any given droplet may be the same as, e.g., 0, 1, 5, 10, 20 or even a hundred or more other droplets in the collection.

Some of the above and certain other aspects of the invention may involve, e.g., protein-protein, protein-nucleic acids, protein-ligand, nucleic acid-nucleic acid, cell-protein, cell-cell, ligand-ligand, ligand-receptor, small molecule-macromolecule, and others. In an aspect disclosed above, for example, the first, second and./or additional known moieties in the droplets in certain exemplary embodiments are proteins and the candidate binding moieties in the droplets are candidate Abs for one or more of such proteins. In certain exemplary embodiments the candidate binding moieties are mAbs and the known moiety is an Ag. Other embodiments also are contemplated in which it is desirable to identify the protein target of an Ab. In such embodiments the target moiety may be the Ab and the candidate binding moieties are a library of proteins. These and other screening applications of the methods, systems and devices disclosed here will be readily apparent to those of ordinary skill in the art given the benefit of this disclosure.

In accordance with another aspect, interaction screening methods, systems and devices are provided for identifying one or more candidate binding moieties based on their ability to bind to a target moiety. The screening comprises providing droplets of liquid medium, where each of the droplets (“each of the droplets” and similar expressions used here meaning each of all, most or at least some of the droplets) contains at least one recombinant binding moiety as a candidate binding moiety and at least one target moiety. The candidate binding moiety of each of the droplets is different from the candidate binding moiety of the other droplets.

The target moiety typically, although not necessarily, is the same in all of the droplets. The recombinant binding moieties distributed amongst the droplets may, e.g., be from a library of such moieties, e.g., from a naïve Ab library, i.e., an unbiased, randomized library that was not made in an animal's immune system for any particular antigen. One such non-limiting subset of such libraries may, e.g., be of single-chained recombinant Fv fragments of antibodies (“scFv”), such antibody fragments being convenient to produce in recombinant systems and which are designed to contain binding regions typical of conventional antibody structures. Naïve scFv libraries can be provided, for example, in accordance with the techniques disclosed in U.S. patent application publication No. 20060160178, the entire disclosure of which is incorporated herein by reference. Optionally the recombinant binding moieties are labeled for subsequent detection, as discussed further below. The candidate binding moiety and target moiety may be any suitable pair of moieties.

In certain exemplary embodiments recombinant binding moieties contained in the droplets as candidate binding moieties are provided by a recombinant microbe in the droplet, for example, yeast, bacteria, e.g., Bacillus subtitilis, E. coli, M13 or other bacteriophage, baculovirus, adenovirus, fungus, etc. As discussed further below, in the methods, systems and devices disclosed here, the candidate binding moiety in a droplet can be secreted or otherwise provided by the recombinant microbe, e.g., presented on the surface of such microbe. In certain exemplary embodiments of the screening methods, systems and devices disclosed here, the candidate binding moieties are provided during incubation of recombinant microbes previously encapsulated in (i.e., provided in) the droplets. In such exemplary embodiments, a collection of recombinant microbes is provided in a culture medium suitable for incubation of the microbes to provide a library of candidate binding moieties. The candidate binding moiety provided by the microbe in each of the droplets is different from the candidate binding moieties provided by the microbes in others of the droplets. That is, as discussed above, the microbe of one droplet would be genetically different from the microbes of other droplets so as to provide a recombinant binding moiety (as a candidate binding moiety for the droplet) correspondingly different from the recombinant binding moiety provided by the microbe within at least most other droplets. Thus, different ones of the microbes in such embodiments can each provide at least one recombinant binding moiety as a candidate binding moiety different from the recombinant binding moiety provided as a candidate binding moiety by most or all others of the microbes in other droplets.

The target moieties optionally may be provided on support particles, e.g., beads or other solid support particles. The droplets optionally are incubated in certain exemplary embodiments, depending on the particular application, and then are examined to identify those droplets, if any, in which the candidate binding moiety is bound to the target moiety. For example, at least a portion of the droplets can be exposed to a detector, e.g., by passing droplets in a single-file stream through a detection site comprising a constricted portion of a microchannel in a module, to identify those in which the candidate binding moiety is bound (or bound with sufficient affinity and/or specificity) to the target moiety. The identified droplets can then be isolated, e.g., by operating a sorter to separate such droplets from other droplets. It should be understood, as mentioned above, that unless otherwise expressly stated, reference here and in the claims to a property or condition of the droplets or of “each of the droplets”, etc., means numerous (e.g., all, most or some) of the droplets, but not necessarily all of the droplets. Similarly, reference to performing an action on the droplets or on each of the droplets means performing such action on numerous (e.g., all, most or some) of the droplets—either collectively or one-by-one, depending on context—but not necessarily performing such action on all of the droplets.

In certain exemplary embodiments of the screening methods, systems and devices disclosed here for identifying one or more candidate binding moieties based on binding to a target moiety, the droplets are provided by forming an emulsion in oil of a dispersion of aqueous droplets, such droplets containing recombinant binding moieties. In certain exemplary embodiments the candidate binding moieties and target moieties are encapsulated into droplets as a stream of droplets, e.g., a single-file stream of droplets in a microfluidic channel. In embodiments, for example, in which the candidate binding moieties are provided by recombinant microbes in the droplets, the microbes can be separated into a stream of droplets of culture medium in a microfluidic channel.

The methods, systems and devices disclosed here further comprise identifying one or more of the droplets, if any, in which the candidate binding moiety has become bound to the target moiety. In certain exemplary embodiments the candidate binding moieties, e.g., recombinant binding moieties, are labeled for visual or other optical detection of binding to the target moiety. All or at least one or more of the identified droplets in which the candidate binding moiety has become bound to the target moiety are then sorted, i.e., segregated or separated from others of the droplets. Further processing of the selected recombinant binding moiety can then proceed, e.g., by further screening, evaluation, production etc., in accordance with any suitable techniques, including techniques currently known or developed in the future.

In certain exemplary embodiments in accordance with this aspect, the droplets further contain a non-target moiety or other known moiety or multiple other known moieties against which the candidate binding moiety is to be screened along with the target moiety. The benefit can be achieved in certain exemplary embodiments having multiple known moieties in each droplet, of improved characterization of the binding properties of the droplet's candidate binding molecule. Thus, for example, certain exemplary embodiments having multiple known moieties in each droplet are suitable for determining (at least preliminarily) binding specificity of the candidate binding moieties for the target moiety. In other words, in these embodiments, the specificity of a candidate binding moiety may be assessed by determining the tendency of that candidate binding moiety to bind to the target moiety along with whether or not it binds to one or more other known moieties acting as non-target moieties. In certain exemplary embodiments it is determined whether the binding affinity of the candidate binding moiety in a particular droplet is greater for the target moiety than it is for one or more non-target moieties. The binding, if any, of the candidate binding moiety to target and non-target moieties can be determined simultaneously or separately, e.g., in separate steps. One or more such non-target moieties in a droplet may be carried on a support particle, e.g., a bead, etc., or can be in the droplet not carried by a support particle. As noted above, the target moiety optionally is carried on one or more particles in a droplet, and similarly the non-target moiety can be carried on one or more other particle(s) in the droplet.

In certain exemplary embodiments, in instances in which it is desirable to identify a binding moiety that binds to two or more structurally related target moieties, the structurally related targets are present along with the candidate binding moiety in a droplet and it is determined whether the candidate binding moiety binds to each of the structurally related targets. The structurally related targets may be any of various classes of molecules, e.g., proteins, polypeptides, peptides, nucleic acids, or other types of organic and inorganic molecules. Non-limiting examples of such structurally related molecules are isomers of small molecules, isoforms of proteins, proteins encoded by members of a gene family or proteins that have and have not undergone various extents of PTM.

In certain exemplary embodiments the binding of a candidate binding moiety is determined for one or more target moieties and for one or more non-target moieties in a droplet, such that a selected candidate binding moiety binds to the one or more target moieties with a higher affinity than it binds to any of the non-target moieties in a droplet. In such embodiments in which a support particle is employed, the support particle itself (i.e., without an attached known moiety) can serve as an example of a “non-target” moiety in order to identify those candidate binding moieties that might be binding to the support particle rather than to a target molecule attached to the support particle.

In certain exemplary embodiments two or more known moieties are attached to the same support particle and it is determined whether a candidate binding moiety binds to one or more of the known entities attached to the support particles. Technologies such as fluorescence resonance energy transfer (FRET) may be used in such embodiments to predict which of the known moieties on the support particle are being bound by the candidate binding moiety.

It should be understood that reference to a droplet containing a known moiety or a target moiety or a non-target moiety means that the droplet contains one or more copies of that moiety. Similarly, reference to a droplet containing a candidate binding moiety means that the droplet contains one or more copies of the candidate binding moiety. Correspondingly, a droplet containing a candidate Ab or a candidate protein, a candidate ligand, etc., means that the droplet contains, respectively, one or more copies of that Ab, protein, ligand, etc.

In accordance with another aspect of the invention, interaction screening methods, systems and devices are provided for identifying one or more candidate binding moieties based on binding to a first known moiety, a second known moiety and optionally one or more additional known moieties, wherein the screening comprises providing droplets of liquid medium as a single-file stream of droplets in a microfluidic channel and determining whether the candidate binding moiety is attached or bound to the first and/or second known moiety, and to the additional moiety(ies), if any. In certain exemplary embodiments this is accomplished using flow-focusing geometry to form the droplets. In accordance with this aspect of the invention, a selection/counter-selection paradigm is implemented in the droplets. In certain exemplary embodiments the droplets each contains the candidate binding moiety and, as the known moieties, at least one target moiety and at least one non-target moiety. A non-target moiety (here, again, consistent with the explanation above, meaning one or more copies of such moiety) is provided in each droplet along with the target moiety and candidate binding moiety. The candidate binding moiety in each droplet in certain exemplary embodiments of this aspect of the invention are recombinant moieties, while in other exemplary embodiments of this aspect the candidate binding moiety is not a recombinant moiety. Detection of the candidate binding moiety of the droplet binding to the non-target moiety in addition to binding to the target moiety provides an indication that the binding of the candidate binding moiety is non-specific for the target moiety, and thus that the candidate binding moiety might be undesirable for a desired purpose. In certain exemplary embodiments the non-target moiety can be provided in the droplet on support particles, e.g., beads or other solid support particles. In certain exemplary embodiments the candidate binding moieties are Abs, or mAbs and the target moiety is an Ag. In such embodiments microfluidics is used to form, identify and/or collect droplets containing the Abs or mAbs that bind to the target Ag to a significantly or sufficiently greater extent than they bind to the non-target moiety. In certain exemplary embodiments the Abs or mAbs are generated against unmodified proteins, post-translationally-modified proteins or other antigens.

### BRIEF DESCRIPTION OF THE DRAWINGS

The methods, systems and devices disclosed here are discussed with reference to the appended drawings in which:

FIGS. 1A and 1B are schematic illustrations of known hybridoma and phage display techniques, respectively, for producing and identifying mAbs to a given Ag.

FIGS. 2 and 2A-2D are schematic illustrations, partially broken away, of devices and systems suitable for implementing microfluidic actions in certain exemplary embodiments of the methods disclosed here, including generating, combining, timing and sorting droplets.

FIG. 3A is a schematic illustration, partially broken away, of a microfluidic device and system suitable for implementing microfluidic actions in certain exemplary embodiments of the methods disclosed here, including generating droplets. FIG. 3B is a graph showing the effect of microfluidic droplet diameter on selected system properties for an exemplary embodiment operating at a 1000 Hz droplet creation rate, specifically the effect on droplet volume and water flow rate.

FIG. 4 is a schematic illustration, partially broken away, of a microfluidic device and system suitable for implementing microfluidic actions in certain exemplary embodiments of the methods disclosed here, including merging or coalescence of droplets flowing singe-file in a microfluidics channel, by applied voltage across electrodes on opposite sides of the channel.

FIG. 5 is a schematic illustration, partially broken away, of a microfluidic device and system suitable for implementing microfluidic actions in certain exemplary embodiments of the methods disclosed here, including passing ordered, coalesced droplets in a serpentine channel of a microfluidics module, e.g., for timed incubating of the droplets, etc.

FIG. 6 is a schematic illustration, partially broken away, of a microfluidic device and system suitable for implementing microfluidic actions in certain exemplary embodiments of the methods disclosed here, including droplet sorting in a microfluidics module.

FIG. 7 is a schematic illustration, partially broken away, of a microfluidic device and system suitable for identification of hybridoma cells secreting mAbs that bind to a target moiety in a microfluidic droplet for subsequent sorting from droplets with mAbs that do not bind to the target moiety.

FIG. 8A is a schematic illustration, partially broken away, of a microfluidic device and system suitable for encapsulation of hybridoma cells in microfluidic droplets in accordance with FIG. 7, and associated graphs, including a graph (on the right side of FIG. 8A) indicating percentage of live vs. dead cells when measured after 7 hours. FIG. 8B is a graph showing cell survival rates.

FIG. 9 is a schematic illustration, partially broken away, of a microfluidic device and system suitable for implementing microfluidic actions in certain exemplary embodiments of the methods disclosed here, including encapsulation of hybridoma cells in microfluidic droplets with two types of beads: one coated with a target Ag and a secondary bead that is not coated with a target Ag.

FIG. 10 is a schematic illustration, partially broken away, of a microfluidic device and system suitable for implementing microfluidic actions in certain exemplary embodiments of the methods disclosed here, including encapsulation in microfluidic droplets of microbes able to secrete protein directly into the culture medium, with two types of beads: one coated with a target Ag and a secondary bead coated not coated with the target Ag.

FIG. 11 is a schematic illustration, partially broken away, of a microfluidic device and system suitable for implementing microfluidic actions in certain other exemplary embodiments of the methods disclosed here, including (i) co-encapsulation in microfluidic droplets of two types of beads, one coated with a target Ag and a secondary bead not coated with a target Ag, together with M13 phage-infected E. coli able to secrete protein directly into the culture medium, (ii) incubation of the droplets, (iii) merging of the droplets with second droplets containing labeled anti-M13 antibodies, and (iv) detecting and sorting of beads to which M13 phage are bound.

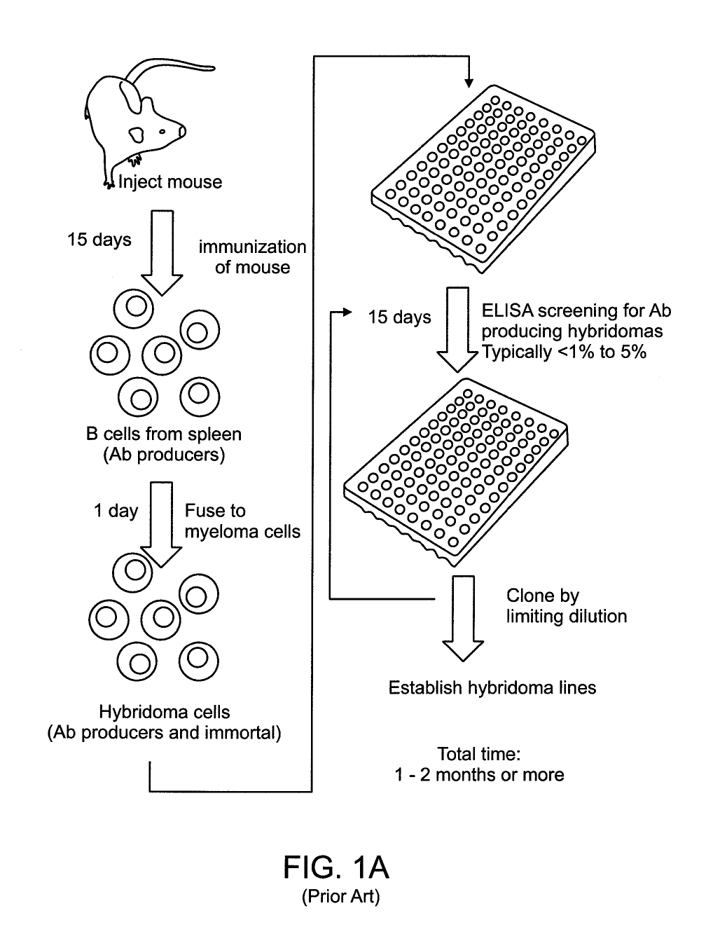

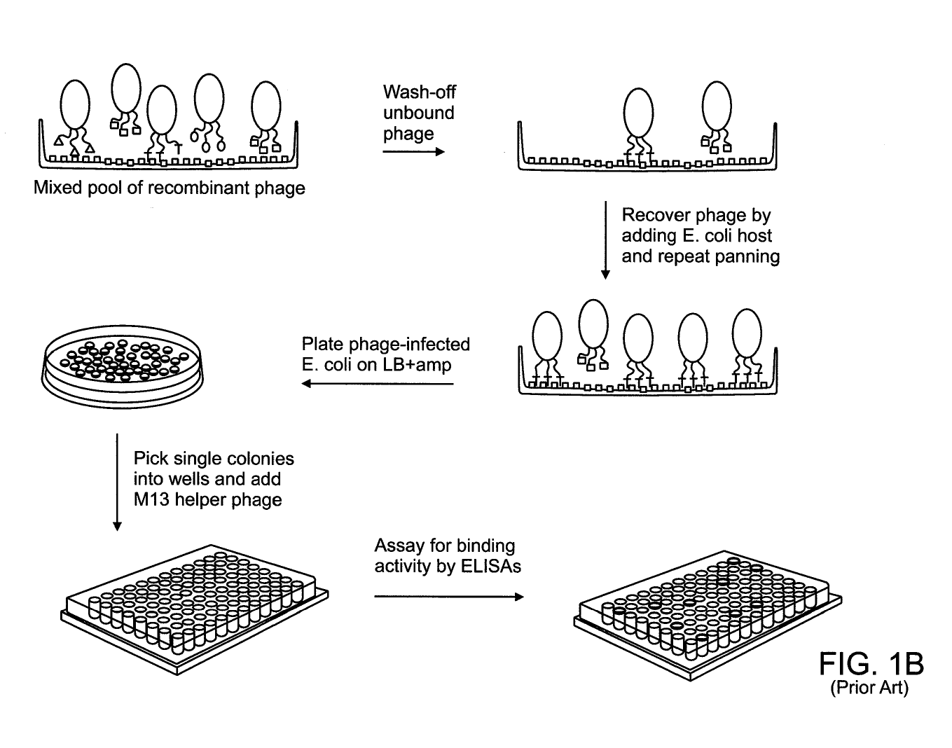

### DETAILED DESCRIPTION OF CERTAIN EXEMPLARY EMBODIMENTS OF THE INVENTION

It will be seen, below, that the following more detailed description of certain exemplary embodiments of the methods, systems and devices disclosed here involve Ab—Ag reactions and the use of microbeads as solid supports for binding targets in the micro-fluidically manipulated droplets. It should be understood, however, that there are numerous other applications and embodiments of the methods and systems disclosed here, where interacting moieties are determined. Certain aspects of the invention may involve, e.g., protein-protein, protein-nucleic acid, protein-ligand, nucleic acid-nucleic acid, cell-protein, cell-cell, ligand-ligand, ligand-receptor, small molecule-macromolecule and others. Numerous such embodiments involving alternative applications of the invention will be readily apparent to those of ordinary skill in the art given the benefit of this disclosure.

From the disclosure here, taken in its entirety, it will be recognized by those skilled in the art, that in the methods, systems and devices disclosed here each such droplet can, to an extent, serve the function of and, so, replace one well in the 96-well plates used in prior techniques. In view of the large number of droplets processable in the methods, systems and devices disclosed here, substantial cost, productivity, efficiency and timing advantages can be achieved in at least certain exemplary embodiments.

In accordance with one aspect of the invention, as disclosed above, certain interaction screening methods, systems and devices for identifying one or more candidate binding moieties based on binding to one or more target moieties comprise providing droplets of liquid medium, at least a majority of which contain at least one recombinant binding moiety as a candidate binding moiety and at least one target moiety. The candidate binding moiety of each of numerous droplets is different from the candidate binding moiety of others of the droplets. One or more of the droplets in which the candidate binding moiety has become bound to the target moiety (if any) are identified and may then be segregated from others of the droplets. In certain exemplary embodiments such interaction screening comprises forming an emulsion in oil of droplets of an aqueous dispersion of recombinant binding moieties. Such emulsion can be formed in any suitable manner, for example, by forming a mixture of the aqueous dispersion and oil and shaking the mixture, stirring the mixture, otherwise agitating or vibrating it or the like.

Alternatively, the droplets can be formed by microfluidic treatment of a liquid medium, e.g., by microfluidic encapsulation. For example, one microfluidic encapsulation technology suitable for at least certain exemplary embodiments uses a flow-focusing geometry in microfluidic channel to form the drops. An aqueous stream is infused through a narrow constriction in the channel. A counter-propagating stream(s) of carrier fluid, e.g., oil, hydrodynamically focus the aqueous stream and stabilize its breakup into substantial uniform, micron-sized droplets as it passes through the constriction. The generation rate, spacing and size of the aqueous droplets is controlled by the relative flow rates of the oil and the aqueous streams and by nozzle geometry. Typical throughput can be 3,000 to 10,000 droplets per second and typical droplet size is uniformly 5 to 100 microns, e.g., about 30 microns. When necessary for a particular application, each of the droplets can be merged or collapsed with one or more secondary droplets. For example, each of the droplets can be merged with a secondary droplet containing additional components come e.g., target moieties, non-target moieties, labeling entities, supplemental culture medium, liquid to alter the properties or condition of the medium, etc. Droplet coalescence can be achieved micro-fluidically, for example, by first interweaving the two droplet types into a single-file stream of alternating droplet types in a microfluidic channel. The sizes of the two droplet populations can be engineered to be different such that they travel along the microfluidic channel at different velocities to achieve droplet pairs. These pairs are then driven to coalesce by, e.g., field-induced dipoles where the droplets pass through an electric field. In certain exemplary embodiments such a microfluidic channel and the electric field generating components are integrated with the droplet-forming microfluidic channel in a common module.

In certain exemplary embodiments the droplets are cultured or otherwise incubated one or more times, as appropriate to the particular application. The droplets can be incubated as a collective mass, e.g., while temporarily held as a collected emulsion in a non-microfluidic container. Alternatively or in addition, the droplets can be incubated as they pass in a microfluidic channel, e.g., in a single file stream. A precise reaction or incubation time can be achieved, e.g., using a timing channel, that is, by passing the droplets at a known flow rate along a microfluidic channel of known length.

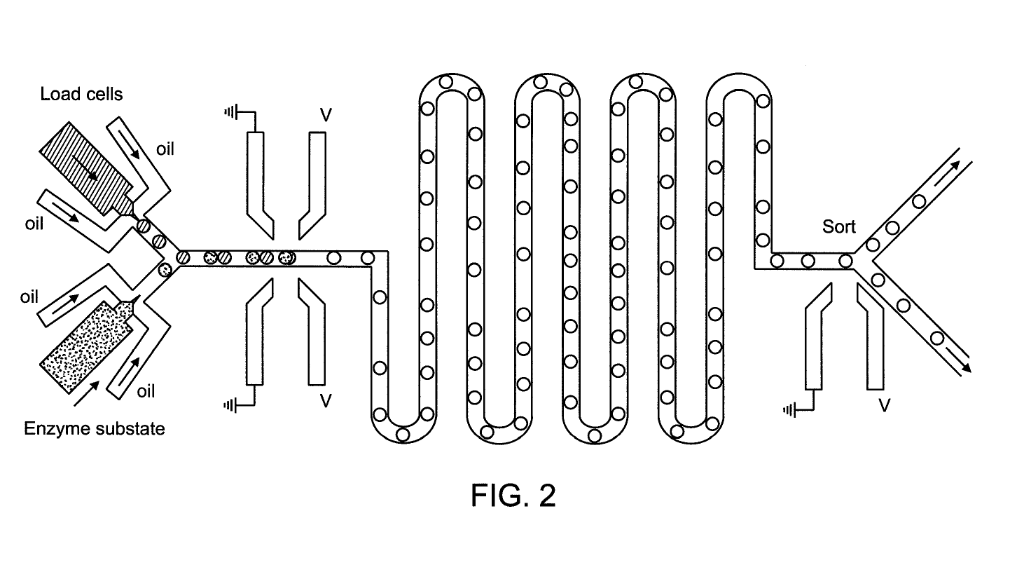

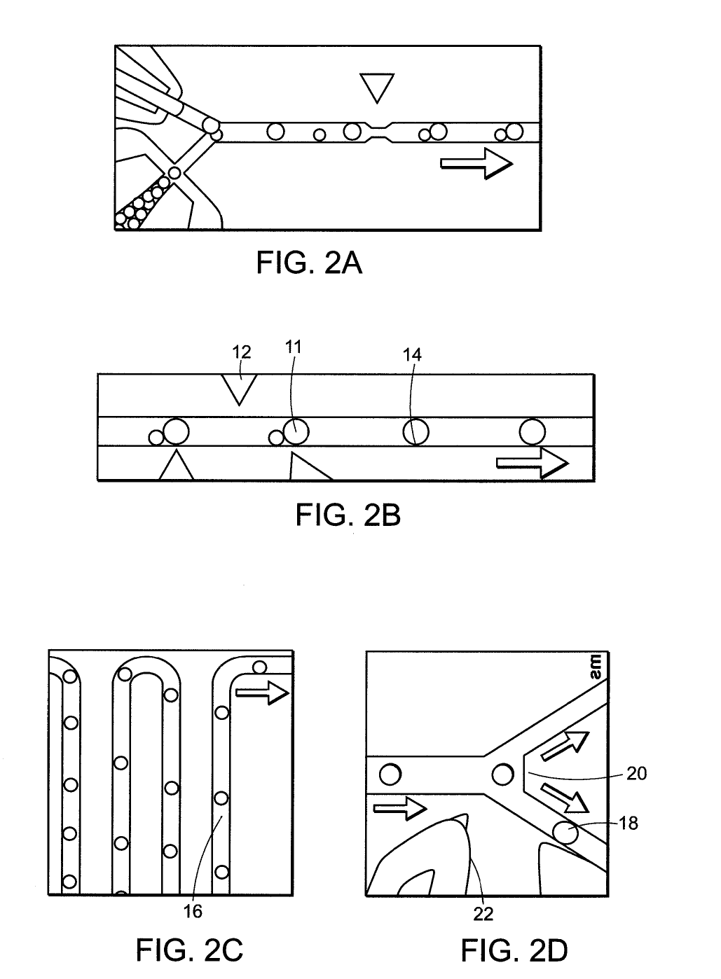

FIG. 2 schematically illustrates technology for generating, evaluating or examining, and/or sorting droplets in a microfluidic module for the methods, systems and devices disclosed here. FIG. 2A shows two different droplet types, having different sizes, being formed and fed in alternating fashion into a common downstream microfluidic channel. In certain exemplary embodiments one of the droplet types contains recombinant binding moieties or microbes able to express and secrete recombinant binding moieties into the droplet medium, while the second droplets contain target moieties, optionally on a substrate, and optionally a non-target moiety, again optionally on a substrate. FIG. 2B shows pairs of droplets in a microfluidic channel in a module 10 or other device in accordance with the present disclosure. The droplet pairs 11 are seen to enter into an electric field generated by electrodes 12, which induces coalescence into a stream of single droplets 14. Either droplet of a droplet pair being merged can be referred to as the supplemental droplet. Such droplet merger techniques can be employed once one more to construct the droplets used in the methods, systems and devices disclosed here, with all of the required components, including, e.g., (depending upon the application and the embodiment) candidate binding moieties or microbes to produce them in the droplet, target moieties, supplemental liquid medium, droplet modifying material, e.g., to alter the pH, salt concentration or other characteristic, property or chemistry of the droplet, etc. Merged single droplets are seen in FIG. 2C to travel in serpentine micro-channel 16 which transports droplets back and forth within a relatively compact or small portion of the module to provide a precise time for a desired event(s) to occur, for example, incubation to populate the droplet with candidate binding moieties from a microbe contained by the droplet, binding to the target moiety, or other event(s) and, optionally, to be tracked and recorded following appropriate signal amplification. FIG. 2D shows droplet segregation. In the embodiment illustrated in FIG. 2D, droplets 18 identified to have a signal or other detected characteristic indicative of the candidate binding moiety of that droplet binding to the target moiety are segregated from others of the droplets at juncture 20 via actuation of electrodes 22. Various implementations of these and other suitable techniques for providing, examining and manipulating the droplets employed in the methods, systems and devices disclosed here will be apparent to those skilled in the art given the benefit of this disclosure.

As noted above, in certain exemplary embodiments the target moiety in a droplet can be carried on a support particle in the droplet. Similarly, in those embodiments where a non-target moiety is employed, the non-target moiety or at least some of the non-target moieties in a droplet can be carried on a support particle in the droplet. The support particle optionally is a solid particle, for example, a bead, such as a micro-bead. In certain exemplary embodiments, including, for example, those wherein the target moieties and, (if used) the non-target moieties are not carried on a support, detection of the droplets in which the candidate binding moiety binds to the target moiety (and/or, as the case may be, to the non-target moiety) can comprise the use of fluorescence polarization screening in accordance with known techniques whose applicability to the method, systems and devices disclosed here will be apparent to those skilled in the art given the benefit of this disclosure.

One type or more than one type of candidate binding moiety can be provided in each of the droplets. Likewise, one type or more than one type of target moiety can be provided in each of the droplets. Similarly, in those embodiments in which a non-target moiety is employed, one or more than one type of non-target moiety can be employed in each droplet.

As disclosed above, in certain exemplary embodiments the recombinant binding moiety in each of the droplets is provided by a microbe in that droplet secreting the recombinant binding moiety. The microbes provide a library of candidate binding moieties in the droplets. The use of micro-organisms has advantages in at least certain exemplary embodiments, including reduced costs, sustained cell viability under microfluidic separation conditions, better cell recovery and ease of handling. For example, Bacillus subtilis can survive for at least 6 days in droplets under aerobic conditions. The microbe may provide the recombinant binding moiety by secreting directly into the medium of the droplet. In certain exemplary embodiments the microbe provides the recombinant binding moiety upon lysing of the microbe in the droplet, e.g., by action of a bacteriophage that defines the recombinant binding moiety, etc. In certain exemplary embodiments the microbes each secretes recombinant binding moieties directly into the liquid medium. The microbe in each droplet provides a candidate binding moiety different from the candidate binding moiety provided by some or all other microbes in other droplets. That is, a genetically different microbe in each droplet provides one or more recombinant binding moieties as the candidate binding moieties for that droplet, such that the recombinant binding moiety(ies) in each droplet is different from some or all of the recombinant binding moiety in the other droplets, corresponding to the genetically engineered differences in the respective microbes. The recombinant binding moieties can be Abs, proteins, nucleic acids, etc. and the liquid medium of the droplets in certain exemplary embodiments is a culture medium for the microbes producing such recombinant binding moieties. In this regard, the droplets can be incubated, e.g., as a collected mass, in a microfluidic channel, as discussed above in connection with FIG. 2, or in any other suitable manner. Given the benefit of this disclosure, those skilled in the art will recognize the applicability of various known techniques for creating recombinant binding moieties in the droplets.

Exemplary embodiments in which a microbe or microorganism in the droplet provides the recombinant binding moiety include, for example, viral, phage or yeast display or bacterial, archea or eukaryote secretion. Non-limiting examples of possible means for encoding, expressing and displaying protein in a droplet include display technology wherein the encoding gene is physically connected to the binding moiety, and secretion technology wherein the binding moiety is secreted from the encoding microbe into the medium, where it is retained within a droplet. Viral and phage display can be used for high-throughput screening of protein interactions using either viral and eukaryote host or bacteriophage and bacterial hosts. In the case of M13 filamentous phage display, for example, the DNA encoding the protein or peptide of interest is ligated into the pIII or pVIII gene. Infected host cells enables packaging of the phage DNA and assembly of the mature virions with the relevant protein fragment as part of their outer coat on either the minor (pIII) or major (pVIII) coat protein. The incorporation of many different DNA fragments into the pIII or pVIII genes generates a library from which members of interest can be isolated. In a typical experiment, by immobilizing a relevant DNA or protein target(s) to the surface of a solid support, a phage that displays a protein that binds to one of those targets on its surface will remain while others are removed by washing. Those that remain can be eluted, used to produce more phage (by bacterial infection with helper phage) and so produce a phage mixture that is enriched with relevant (i.e. binding) phage. The repeated cycling of these steps is referred to as ‘panning’, in reference to the enrichment of a sample of gold by removing undesirable materials. Phage eluted in the final step can be used to infect a suitable bacterial host, from which the phagemids can be collected and the relevant DNA sequence excised and sequenced to identify the relevant, interacting proteins or protein fragments. Also, yeast display (or yeast surface display can be used. A protein of interest is displayed as a fusion with, e.g., the Aga2p protein on the surface of yeast. The Aga2p protein is naturally used by yeast to mediate cell-cell contacts during yeast cell mating. As such, display of a protein via Aga2p projects the protein away from the cell surface, minimizing potential interactions with other molecules on the yeast cell wall. The use of magnetic separation and flow cytometry in conjunction with a yeast display library is a method to isolate high affinity protein ligands against nearly any receptor through directed evolution. Bacterial display (or bacteria display or bacterial surface display) is a protein engineering technique used for in vitro protein evolution. Libraries of polypeptides displayed on the surface of bacteria can be screened using flow cytometry or iterative selection procedures (biopanning). Typical fusions in bacteria such as E. coli include the flagellin protein.

In addition, eukaryote secretion can be used for encoding, expressing and displaying protein in a droplet. In all eukaryotic cells, there is a highly evolved process of secretion. Proteins targeted for the outside are synthesized by ribosomes docked to the rough endoplasmic reticulum. As they are synthesized, these proteins translocate into the ER lumen, where they are glycosylated and where molecular chaperones aid protein folding. Misfolded proteins are usually identified here and retrotranslocated by ER-associated degradation to the cytosol, where they are degraded by a proteasome. The vesicles containing the properly-folded proteins then enter the Golgi apparatus. In the Golgi apparatus, the glycosylation of the proteins is modified and further posttranslational modifications, including cleavage and functionalization, may occur. The proteins are then moved into secretory vesicles which travel along the cytoskeleton to the edge of the cell. More modification can occur in the secretory vesicles (for example insulin is cleaved from proinsulin in the secretory vesicles). Eventually, the vesicle fuses with the cell membrane at a structure called the porosome, in a process called exocytosis, dumping its contents out of the cell's environment.

In addition, bacterial and archea secretion can be used for encoding, expressing and displaying protein in a droplet. Secretion is well-known in bacteria and archaea. The Sec system is a conserved secretion system which is homologous to the translocon in the eukaryotic endoplasmic reticulum consisting of Sec 61 translocon complex in yeast and Sec Y-E-G complex in bacteria. Secretion via the Sec pathway generally requires the presence of an N-terminal signal peptide on the secreted protein. Gram-negative bacteria have two membranes, thus making secretion topologically more complex. So there are at least six specialized secretion system in Gram-negative bacteria. In Gram-positive organisms, secretion of proteins past the inner membrane allows for their release directly into the medium.

Droplets containing microbes for use in certain exemplary embodiments of the methods, systems and devices disclosed here can be formed, for example, by providing a collection of microbes in a liquid medium, e.g., a culture medium suitable for incubation of the microbes. The droplets can be formed by separating the liquid medium containing the microbe collection or library into a stream of droplets of the medium, e.g., in accordance with any of the various emulsification or microfluidic techniques mentioned above or by other suitable technique. Target moieties can be provided in the droplets by any suitable technique, e.g., by droplet merger techniques disclosed above. When appropriate for the particular application, the droplets can be incubated, e.g., in a collected mass, in a single-file stream in a microfluidic channel or in any other suitable manner.

In certain exemplary embodiments the microbes secrete into the medium of their respective droplets recombinant binding moieties which are labeled, e.g., moieties which are labeled with green fluorescent protein (GFP). Numerous suitable alternative labeling for the recombinant binding moieties expressed by the microbes included in the droplets will be apparent to those skilled in the art given the benefit of this disclosure. The label provides a detectable marker for the candidate binding moieties and may be directly present, as in the case, e.g., of GFP-fusion proteins, or it can be conditionally present, as in the case, e.g., of a secondary detectable label that can be attached to the candidate binding moiety subject to an event occurring subsequent to its production, e.g., upon binding to the target moiety. One of numerous non-limiting examples of such secondary or conditional label is a detectably-labeled goat antibody (e.g., raised against human IgG) used to bind to and thereby label a human antibody.

In certain exemplary embodiments the microbes are fungi, e.g., fungi that are members of the genus Pichia or a member of the genus Saccharomyces. In certain exemplary embodiments the microbes are bacteria. Numerous suitable fungi, bacteria and other microbes for use in the methods, systems and devices disclosed here will be apparent to those skilled in the art given the benefit of this disclosure. In at least certain exemplary embodiments, bacterial microbes employed in the droplets to provide recombinant binding moieties and candidate binding moieties for the target moiety are gram-positive bacteria, e.g., Bacillus subtilis. In certain exemplary embodiments Bacillus subtilis are incubated in their respective droplets to secrete directly into a culture medium candidate binding moieties which are scFv-GFP fusion proteins. Collectively among the droplets a library is formed of scFv-GFP fusion proteins as candidate binding moieties, e.g., in certain exemplary embodiments a library of from 10⁶ to 10⁹ different scFv-GFP fusion proteins. In certain exemplary embodiments a naïve scFv library is constructed and cloned into a Bacillus subtilis-E. coli bifunctional plasmid encoding chloramphenical resistance, e.g., into a pC194 derivative. In certain exemplary embodiments a naïve scFv library is constructed and cloned into a suitable microbe in which the Ab gene is flanked by a leader sequence and a reporter gene. For example, in certain exemplary embodiments the Ab genes are cloned 3′ downstream of an amylase leader peptide and 5′ to GFP, resulting in a fusion protein construct in the form: (amino terminus) leader peptide-Ab-GFP protein (carboxyl terminus).

In certain exemplary embodiments the candidate binding moieties are candidate Abs in the form of fusion protein constructs containing a reporter gene, e.g., an enzyme. The reporter gene can be, e.g., β-Galactosidase and the candidate binding moieties can be candidate mAbs in the form: (amino terminus) leader peptide-mAb-β-Galactosidase (carboxyl terminus). In alternative embodiments the reporter gene can be alkaline phosphatase such that the candidate binding moieties are candidate antibodies in the form: (amino terminus) leader peptide-mAb-alkaline phosphatase (carboxyl terminus).

In certain exemplary embodiments, a microbe collection is provided by constructing a naïve scFv library and cloning the naïve scFv library into a bacterium, e.g., a Bacillus subtilis-E. coli bifunctional plasmid. Optionally the hybrid genes in the Bacillus are placed under control of a late spore promoter, e.g., the late spore promoter spoV. In certain exemplary embodiments the clones are transformed into, and expressed in, a strain of Bacillus subtilis, e.g., YB886. The Ab genes can be first cloned into E. coli and plasmid DNA clones then used to transform the Bacillus. Cell growth optionally is done on a solid medium to inhibit biased outgrowth of a limited number of clones. In certain exemplary embodiments the candidate binding moieties are mAbs and the target moiety is an Ag. Optionally, microfluidics are used to form, identify and/or collect droplets containing mAbs that bind to the target Ag to a significantly or sufficiently greater extent than they bind to one or more non-target moiety(ies). In certain exemplary embodiments the mAbs are generated against unmodified proteins, post-translationally-modified proteins or other Ags.

Certain exemplary embodiments employ Bacillus subtilis secreting a scFv-GFP fusion protein. Binding is determined by detection and measurement of the binding of the fusion protein bound to a bead coated with a phosphorylated peptide. Certain exemplary embodiments employ Bacillus subtilis secreting a scFv-GFP fusion protein, and specific binding is determined by measuring the GFP of the fusion protein bound to a bead coated with a phosphorylated peptide and to a second bead-type coated with the unphosphorylated peptide.

Droplets in which the corresponding candidate binding moiety is bound to the target moiety can be identified, i.e., detected amongst and discriminated from those in which binding to the target moiety was not detected by any suitable technique (or, in certain exemplary embodiments, was detectable but deemed to have occurred with insufficient affinity or specificity). The applicability of various available droplet identification techniques in the method, systems and devices disclosed here will be apparent to those skilled in the art given the benefit of this disclosure. For example, droplet identification can be performed by exposing the droplets (i.e., by exposing at least a portion of the droplets) to a detector, and operating the detector to detect droplets, if any, in which the candidate binding moiety is bound (meaning bound at all or bound sufficiently, depending upon the particular application and embodiment) to the one or more target moieties in the droplet. As noted above, in certain exemplary embodiments this will involve identifying one or more beads or other support particles in the droplet carrying the target moiety with which the candidate binding moiety has reacted. For example, laser sensors or other optical sensors at a droplet identification site in a module can be employed to interrogate each droplet as it passes along a microfluidic channel, e.g., single-file through a construction in the channel. In certain exemplary of embodiments in which the recombinant binding moiety provided by the microbe is optically labeled and multiple of the target moieties, e.g., 2-50 target moieties, are carried on a microbead in the droplet, detection can be accomplished by examining each droplet to determine whether or not multiple labels occur in close proximity, that is, in closer proximity than occurs elsewhere in the droplet through normal distribution of the recombinant binding moieties in the liquid of the droplet. This would indicate that the labels have become associated with the particle through binding of the candidate binding moiety to the target moieties carried by the particle. Additional suitable techniques will be apparent to those skilled in the art given the benefit of this disclosure.

Droplets identified in the identification step can then be sorted or isolated from the other droplets. The applicability of known sorting techniques suitable to the methods, systems and devices disclosed here will be apparent to those skilled in the art given the benefit of this disclosure. In certain exemplary embodiments the droplets are collected and a detector is moved past the droplets (or past the droplet's beads or other support particles, if used). Molecular tweezers or molecular clips can be used to collect the appropriate droplets. In other embodiments the droplets (or particles) are moved past the detector. For example, droplets flowing in a single-file stream from an identification site mentioned above in a microfluidic module, can be passed still as a single-file stream in the microfluidic channel to a sorter site in the same module. The sorter site can comprise, for example, a juncture at which the microfluidic channel forks into first and second downstream channels. Droplets identified as having target binding can be passed to the first downstream channel while those not identified as having target binding can be passed to the second downstream channel, thereby segregating or separating potentially relevant droplets from seemingly irrelevant droplets. For this purpose, where the droplets are flowed past the sorter, the sorter can employ any suitable technique for controlling the position or direction of flow of the selected droplets, including, e.g., electrophoresis, electro-wetting, pumping and valves, etc.

FIG. 6 schematically illustrates droplet sorting in a microfluidics module in accordance with certain exemplary embodiments. Droplets, which may be electrically neutral, flow from bottom to top in FIG. 6. Selected droplets are carried passively to one side of a “Y”-shaped bifurcation in the microchannel while the other droplets are pulled to the other side in response to application of a high voltage, high frequency signal to the pair of electrodes 18. In certain exemplary embodiments the droplets can be sorted by forming a one-thick film of the droplets on a surface, followed by identifying those droplets in the film, if any, in which the candidate binding moiety is bound to the target moiety (e.g., to one or more solid support particles carrying target moieties in the droplet). Identification of such droplets can be carried out optically, e.g., by acquiring an optical image of the film and examining the image to determine the X-Y location of the successful droplets. The X-Y location of the identified droplets can be used with any suitable X-Y picker device to collect the identified droplets from the film. In certain exemplary embodiments laser capture dissection is used to pick out the identified droplets from the film.

As noted above, the target moieties can be provided on support particles in the droplets, e.g., beads. Suitable beads for use in the method, systems and devices disclosed here will be readily apparent to those skilled in the art given the benefit of the disclosure. Beads suitable for at least certain applications include, e.g., agarose beads, polystyrene beads, e.g., nickel chelate beads, etc., having an average diameter of, e.g., 1 micron to 30 microns. In certain exemplary embodiments the target moieties are carried by the support particles by covalent bonding. Alternatively, target moieties may be attached to the support particles non-covalently. Any suitable number of target moieties may be carried by a support particle. In certain exemplary embodiments the one or more support particles in a droplet each carries at least 20 target moieties, e.g., 20-500 target moieties. Certain exemplary embodiments employ 200-500 target moieties per bead.

As noted above, in certain exemplary embodiments of the methods, systems and devices disclosed here, non-target moieties are included in the droplets. Such non-target moieties can be used in certain applications to determine binding affinity or specificity of the candidate binding moiety for the target moiety. Depending upon the needs of a particular application, the candidate binding moiety contained in a droplet may be determined to be selectively bound to the target moiety if the candidate binding moiety is bound only to the target moiety or, in other cases, if it is bound to the target moiety sufficiently preferentially versus the non-target moiety. For example, in certain exemplary embodiments the candidate binding moiety in the droplet binds preferentially to the target moiety if the difference in amount or degree of binding is detectable by the particular embodiment of the method, system or device employed. In certain exemplary embodiments the candidate binding moiety may be taken to bind specifically to the target moiety in the detected differential is greater than a selected threshold amount.

The non-target moieties optionally are carried on support particles in the droplet, e.g. beads, especially where the target moieties in the droplet are carried by support particles. The non-target moieties typically may be carried on one or more such support particles in the droplet by covalent bonding or other suitable means which will be apparent to those skilled in the art given the benefit of this disclosure. In certain exemplary embodiments, depending upon the particular application, the non-target moiety may be selected from suitable proteins, protein epitopes, small molecules, modified peptides, cells or viruses, nucleic acids, etc. In certain exemplary embodiments the non-target moieties are simply the surface of support particles of the type carrying the target moieties. That is, the support particles are used as naked beads to determine whether the candidate binding moieties are binding to the surface of the support particles rather than to the target moieties carried by the particles. More generally, beads or other particles similar to or the same as those described above for carrying the target moieties may be used to carry non-target moieties in a droplet. In certain exemplary embodiments, however, the support particles carrying non-target moieties in a droplet are distinguishable from particles carrying target moieties in the droplet. For example, the particles carrying target moieties may be distinguishable from the particles carrying non-target moieties by one or a combination of only one particle having a detectable dye, one particle or the other being a distinguishable quantum dot or being associated with a distinguishable quantum dot, particle types being distinguishable by fluorescence polarization or size, etc. For example, the particles may be optically distinguishable, e.g. each may have a different fluorescence wavelength upon excitation. Accordingly, where the candidate binding moieties are labeled for optical detection or observation, optical interrogation of droplets in such embodiments can distinguish those droplets in which the labeled candidate binding moieties have become bound only to target moieties from those droplets in which the labeled candidate binding moieties are non-specific for the target moiety, having reacted with both the target moieties and the non-target moieties. In the latter droplets both particle types will be observed to be associated with the optically detectable labels. In certain exemplary embodiments, depending upon the needs of the particular application, the non-target moieties may be carried on the second particles in number or density exactly or approximately the same as that of the target moieties are carried on the first particles.

In certain exemplary embodiments each of the droplets contains both target moieties and non-target moieties, neither of which is carried on support particles. In such embodiments, fluorescence polarization can be used in accordance with known techniques to determine whether the candidate binding moieties of a droplet bind to the target moieties, the non-target moieties, neither, or both. Given the benefit of this disclosure, it will be within the ability of those skilled in the art to employ fluorescence polarization in the methods, systems and devices disclosed here. Such determination, in turn, can be employed in then sorting the droplets in accordance with the principles discussed above.

It should be understood that reference to a droplet containing a support particle carrying a target moiety means that the droplet contains one or more such particles. Thus, the support particle may be a single bead carrying one or more of the target moiety(ies), or it may be multiple particles in the droplet, each carrying one or more of the target moiety(ies). In certain exemplary embodiments the support particle carrying a target moiety is a bead having at least 20, e.g, 20-500, typically 200 to 500 of a target Ag for which an Ab is sought, i.e., to which a candidate Ab (the “candidate binding moiety”) in the droplet may or may not be reactive. Similarly, reference here and in the claims to a droplet containing a support particle carrying a non-target moiety (optionally referred to as a decoy particle) means that the droplet contains one or more particles each carrying one or more copies of the non-target moiety. In certain exemplary embodiments the non-target entity is a potential reaction site of the particle itself, such as the particle's own surface, e.g., the surface of an uncoated or “naked” bead of the same type used for the first support particle carrying the target moiety. In certain exemplary embodiments the support particle carrying a non-target entity is a bead having at least 20 of a non-target Ag, e.g, 20-500, typically 200 to 500.

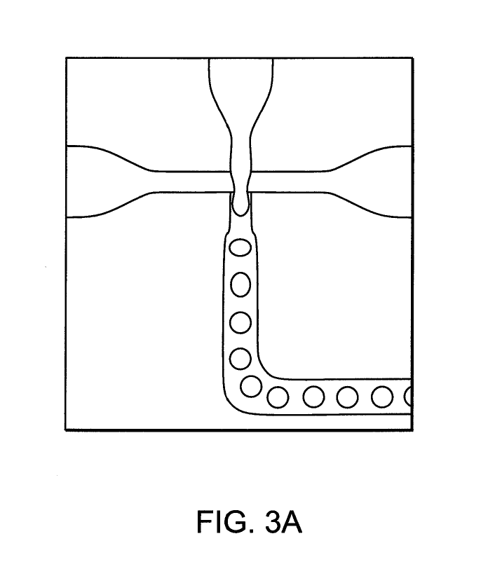

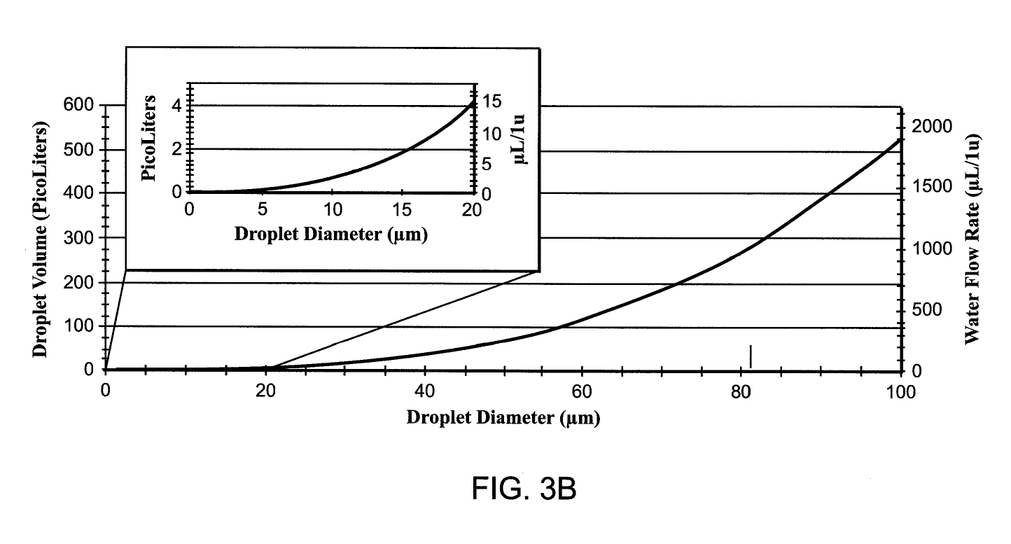

As noted above, certain embodiments of the methods, systems and devices disclosed here employ microfluidic channels, junctions and the like. Certain exemplary embodiments employ networks of microfluidic channels in a module as a flexible platform for the precise manipulation of the droplets. The assembly of one or more such liquid-handling modules into systems provides a convenient and robust implementation methods, systems and devices disclosed here, e.g., in the form of microfluidic chips. One suitable droplet encapsulation technology uses a flow-focusing geometry. An aqueous stream is infused through a narrow constriction in a microfluidic channel. One or more counter-propagating streams of carrier fluid hydrodynamically focus the aqueous stream and stabilize its breakup into micron-sized droplets as it passes through the constriction. Oil is one non-limiting example of a suitable carrier fluid for at least certain exemplary embodiments, depending on the particular application. The generation rate, spacing and size of the aqueous droplets is controlled by the relative flow rates of the oil and the aqueous streams and by nozzle geometry. Typical throughput is 3,000 to 10,000 droplets per second. Droplet generation can be performed in a loading module. Specifically, in such embodiments droplets are formed at the nozzle where one or more carrier fluid streams and a reagent stream are brought together. FIG. 3A is a schematic illustration, partially broken away, of a microfluidic device and system suitable for implementing microfluidic actions in certain exemplary embodiments of the methods disclosed here, including generating droplets. FIG. 3B is a graph showing the effect of microfluidic droplet diameter on selected system properties for an exemplary embodiment operating at a 1000 Hz droplet creation rate, specifically the effect on droplet volume and water flow rate. Variations in nozzle geometry and flow rates can result, in certain exemplary embodiments, in droplets of from 5 μm to 80 μm in diameter and droplet generation rates of from 1,000 Hz to 20,000 Hz. Cells, beads, proteins, nucleic acids and other biomaterials can be encapsulated in such droplets using the loading module. In addition to inducing breakup of the aqueous stream into droplets, the carrier fluid (e.g., oil) also can control the spacing of droplets, referred to here as the duty cycle. A low oil-to-aqueous flow rate ratio typically creates closely spaced droplets and a high duty cycle, while a high oil-to-aqueous ratio spreads droplets apart and decreases the duty cycle. Different duty cycles are desirable for different downstream modules. For example, redirecting droplets requires, in at least certain exemplary embodiments, a minimum spacing of about five droplet diameters, while long on-device delay times or emulsion generation may be more efficient with closely spaced droplets, e.g., less than 2 diameter separation between droplets.

Such embodiments can provide the droplets, each as a well defined, encapsulated microenvironment that eliminates cross contamination or changes in concentration due to diffusion or surface interactions. Droplets provide a good microcapsule that can isolate reactive materials, proteins, cells, or small particles (e.g., beads) for further manipulation and study. In certain exemplary embodiments droplets are passed through microfluidic chips that both sort and recover successful droplets, e.g., droplets in which the candidate binding moiety binds to the target moiety. For example, droplets containing beads can be passed through a channel pinch, that is, a localized narrowing of a microfluidic flow channel, in order to analyze the amount of label on the bead relative to the amount in solution elsewhere in the droplet. In certain exemplary embodiments as few as approximately 1000 fluorescein label molecules on a micro-bead can be detected against the background of a droplet containing a solution of fluorescein.

When necessary for an application, droplet merger or coalescence can be achieved, e.g., by first interweaving the two droplet types, i.e., forming a stream of alternating droplet types. The sizes of the two droplets populations can be engineered to be different such that they travel at different velocities to achieve droplet pairs. These pairs are then driven to coalesce, e.g., by field-induced dipoles created d when the droplet pairs pass through an electric field. A suitable electric field can be created across a microchannel, e.g., at a coalescence site in a microfluidic module, by energizing electrodes at positioned at the coalescence site. A precise reaction time can be achieved, e.g., by using a timing channel of fixed length in the module, as discussed above with reference to FIG. 2A-D.

The droplet sorter of the microfluidic droplet reaction systems disclosed here can sort droplets in microfluidic devices in any suitable manner, for example, through the use of mechanical valves (e.g., Fluidigm). The use of dielectrostatic forces in an electric field gradient provides an alternate means that can be precisely controlled, can be switched at high frequencies, and requires no moving parts. Hence, drop-by-drop sorting based on modulating the application of an external electric field gradient can be readily achieved. A small electric field applied at a bifurcation or fork or junction in a microfluidic channel can precisely dictate which channel a given droplet enters. The large forces that can be imparted on the droplets and the short time required to apply the field make this a fast and robust sorting engine with no moving parts. The processing rate in certain exemplary embodiments is limited only by the rate of droplet generation and electric field switching time, and can readily exceed 10,000 per second.

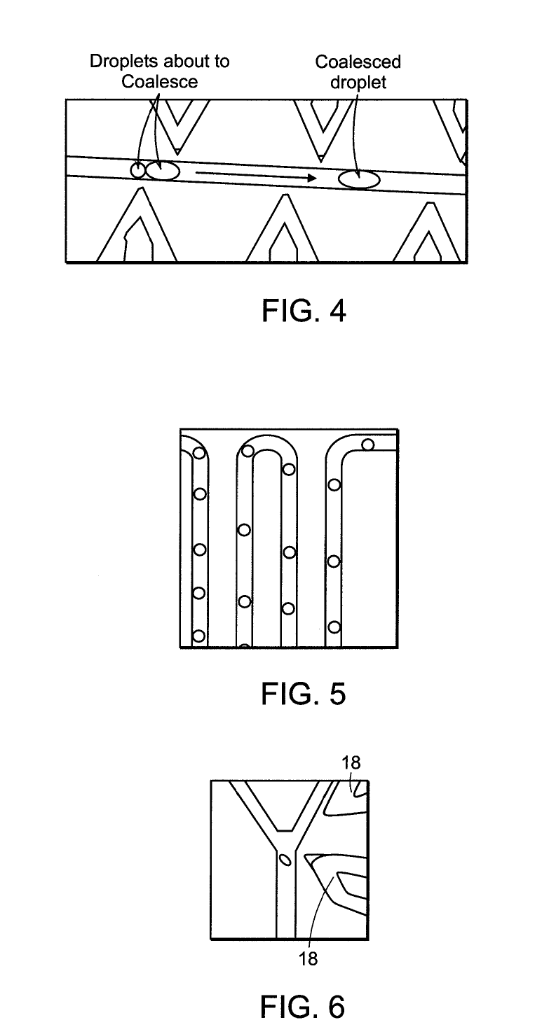

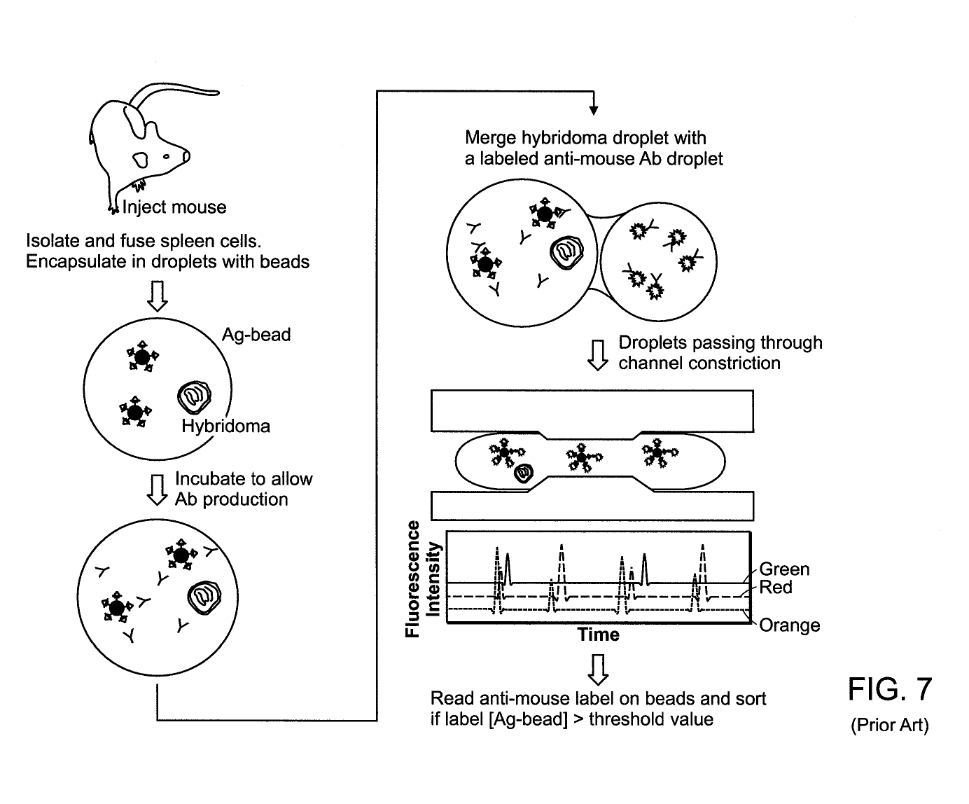

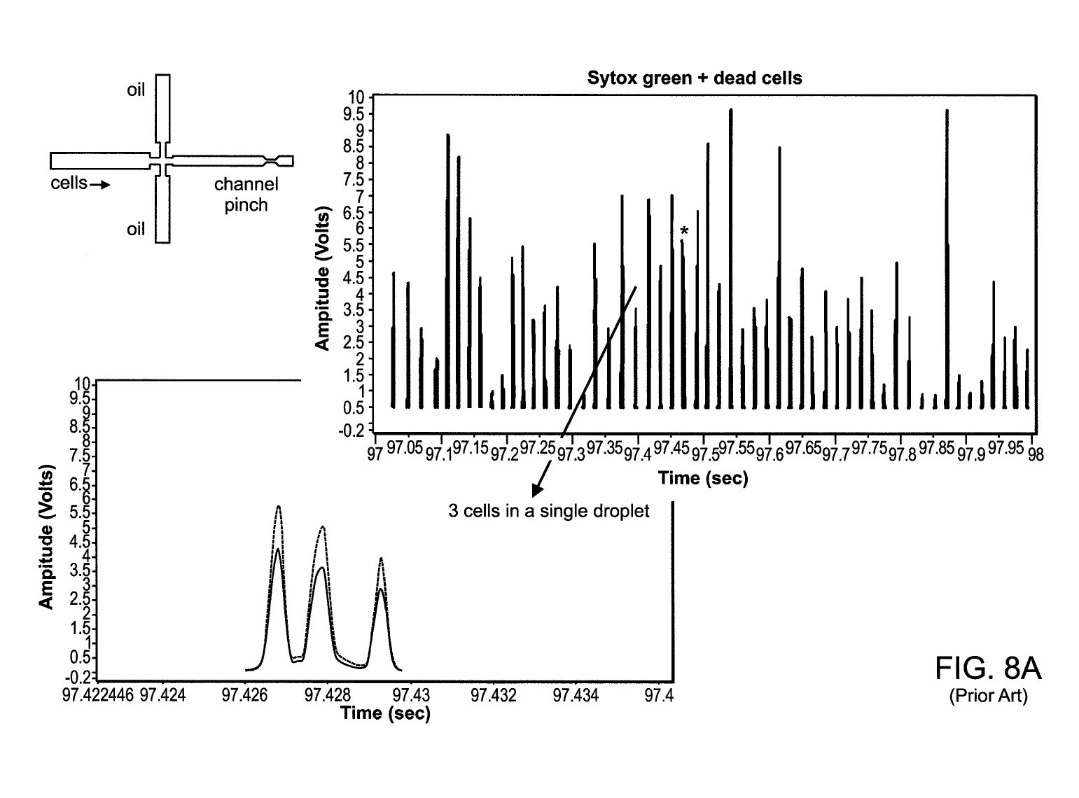

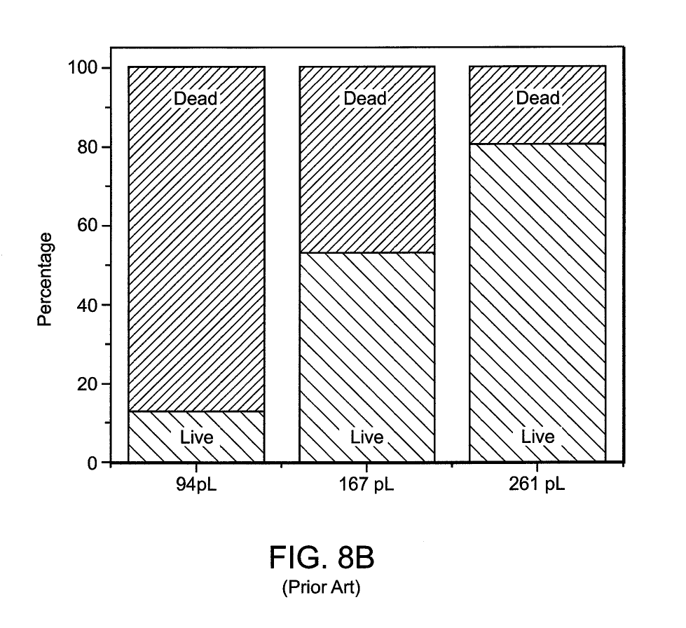

Droplet storage is employed in certain exemplary embodiments. Collected emulsions can be stored for periods of time, e.g., ranging from less than one hour to many months. Emulsion storage makes it possible to incubate droplets, e.g., for a time period required for reactions or transformations in certain microbe based methods, systems and devices. Off-device emulsion storage is employed in certain exemplary embodiments for high-throughput screening of large libraries. The individual entities in the library can be tagged, emulsified, and combined with all the other elements of the library without the fear of cross contamination for long periods of time. These emulsified libraries can be re-injected into a microfluidics device where the individual droplets optionally can be merged, examined, sorted and/or otherwise further processed. As noted above, cell merger can be performed with secondary droplets formed on-device, which secondary droplets may contain, e.g., reagents, cells, support particles, etc. or any combination of such components. In certain exemplary embodiments the methods, systems and devices disclosed here can perform independent assays with picoliter-volume droplet “reactors.” Droplet coalescence or merger of adjacent droplets can be achieved, for example, by interleaving streams containing droplets of two different sizes or viscosities. Smaller (or less viscous) droplets move down channels faster than larger (or more viscous) droplets, causing the small droplets to overtake the large ones. An electric field can be applied continuously or intermittently by actuation of electrodes placed along the channel, inducing the two drops to merge. In FIG. 4, coalescence of droplets is shown within a microfluidics device. Droplets are flowing from left to right in the illustration. The smaller droplets are initially separated from the larger ones, but after traveling some distance down the channel they catch up to their partners (as shown on the left). The pair coalesces, as shown on the right, when a voltage is applied across the electrodes. The electrodes are shown as white triangles in the figure. Various alternative techniques for droplet merger will be apparent to those of ordinary skill in the art given the benefit of this disclosure.

As noted above, certain exemplary embodiments of the methods, systems and devices disclosed here may employ incubation. An incubation or delay site, referred to here also as a delay module, can be used to allow incubation and reaction of droplet contents in a controlled process. Multiple modules can be used in a single chip, e.g., to accommodate multistage screening and assay protocols requiring different incubation conditions for each step. On-chip incubation times can be set by the total flow rate into the delay line, the cross sectional area of the channel, and the length of the line. Perfect ordering (i.e., first in, first out) can be accomplished by keeping the channel cross section similar to the droplet cross section, so as to maintain single-file droplet flow in the channel, as seen in FIG. 5. This prevents droplets from passing each other in the line. Typically, such embodiments will be limited to relatively short transit times, e.g., less than about 30 seconds, due to the high pressure drop caused by the relatively small channels. The microfluidic device and system illustrated in FIG. 5 are suitable for implementing microfluidic actions in certain exemplary embodiments, including passing ordered, coalesced droplets in a serpentine channel of a microfluidics module, e.g., for timed incubating of the droplets, etc. Longer incubation times can be achieved in some cases by making the channel cross section slightly larger than the droplet diameter, resulting in “almost perfect” ordering of the droplets. The variation of the transit time through the delay line preferably is kept to less than 5% in typical embodiments due to the strong affinity for droplets to stick together as they move through the device. Transit times from 30 seconds to 30 minutes are possible with a single layer device, while multi-layer stacking of lines make it possible to increase delay times to 4 hours.

Droplet sorting in a microfluidics module is illustrated in FIG. 6, discussed above. Directing or redirecting droplets at a juncture in a microfluidic channel can be achieved in certain exemplary embodiments by applying an electric field to each droplet just upstream of the fork. The droplets may, for example, be electrically charged and driven by electrophoretic forces or neutral and driven by dielectrophoretic forces. An appropriate field can be generated and applied by means of integrated electrodes in a module. Coupled to a detection site or module (e.g., employing fluorescence detection, backscatter detection, etc.), the sorting module can sort the droplets by comparing the detected results to specified criteria at rates of up to 4,000 droplets per second in certain exemplary embodiments. For example, the droplets may simply be sorted to “keep” and “discard” bins, or to further downstream processing. Appropriate applications will be apparent to those skilled in the art, given the benefit of this disclosure, in which such sorting modules can provide a fast and precise way to remove individual or sub-populations of droplets from the collection of droplets.

Beads or other support particles or the like can be used in certain exemplary embodiments, as noted above, as substrata to which any of various molecules can be attached, e.g., target moieties or non-target moieties. As one non-limiting example, sets of dye-colored beads can be employed, e.g., dye colored beads commercially available from Luminex Corporation (sold for use in multiplex reactions). Suitable materials and techniques are described, for example, in U.S. Pat. No. 6,929,859 issued Jul. 3, 2003 and titled “Precision fluorescently dyed particles and methods of making and using same,” naming as inventors Don J. Chandler, Van S. Chandler, Beth A. Lambert, Janet J. Reber, and Stacie L. Phipps. The entire disclosure of U.S. Pat. No. 6,929,859 is incorporated herein by reference. U.S. Pat. No. 6,929,859 describes techniques for precisely dyeing polystyrene beads of sizes ranging from approximately 10 nm to 100 um in diameter. The bead particles are comprised of material that is water-insoluble but soluble in some suitable solvent. The dyes employed are preferably squaric acid-based molecules that exhibit fluorescence extending into near infrared and/or infrared regions, i.e., to ca. 1,000 nm. The Luminex technology allows for a highly reproducible process in which two or more dyes of independent concentration are absorbed uniformly into each bead, resulting in multiple fluorescent signals characteristic of the dyes present in the bead. For example, to make two or more populations of beads with different fluorescent characteristics, the ratio of red:orange dyes is altered for each population by an adequate increment in proportion of the dyes so that one population does not optically overlap with any other population.

In certain exemplary embodiments a bead map is made containing subset populations of beads, such that there is a discrete distribution of one or more characteristics for each subset population of beads within boundaries prescribed by each region in the bead map. Such characteristics can include, as non-limiting examples, fluorescence polarization, fluorescence resonance energy transfer, fluorescence quenching, pH sensitivity, and size. Two parameters, namely, fluorescent color and color intensity or brightness (expressed in fluorescence channel units) are typically used to establish fluorescence characteristics; bead populations can be classified based on these parameters to generate a “fluorescence signal”. Thus, beads differing in fluorescence signal within a sample, for example a microfluidic droplet, can each be analyzed for their fluorescence signal and thereby assigned to a given location in a bead map.

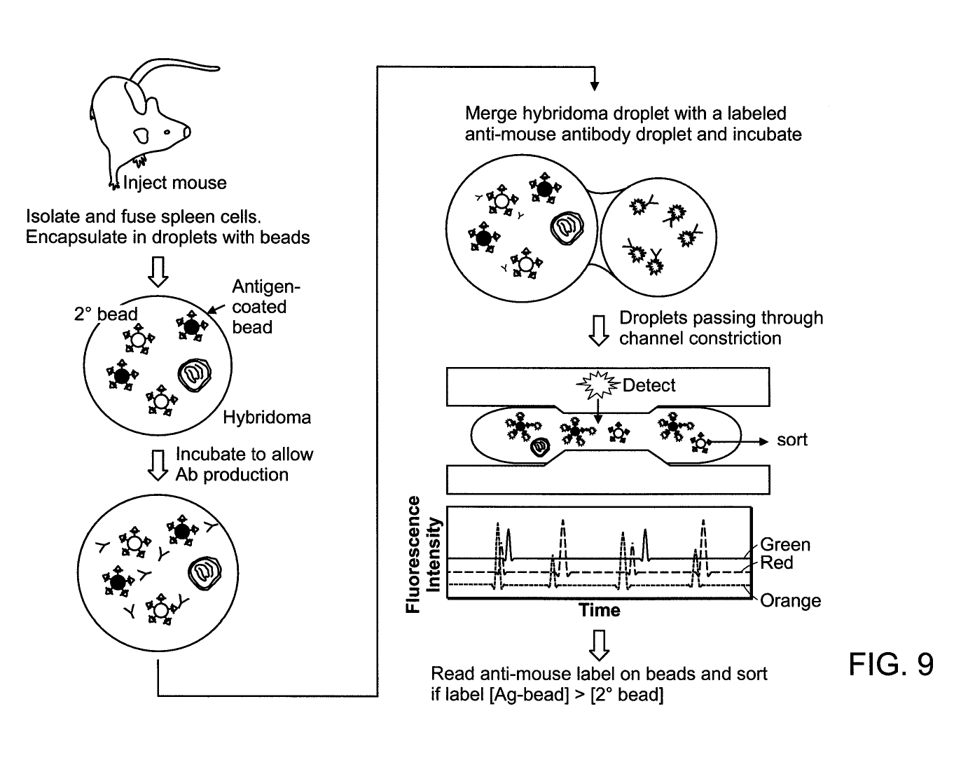

A significant advantage of at least certain exemplary embodiments of the methods, systems and devices according to this aspect of the invention is the ability to rapidly differentiate by microfluidics candidate binding moieties, e.g., mAbs, that bind selectively to their target Ag from those that do not. That is, the process is designed to not only identify Abs that bind to a target Ag, but also ensure that the selected Abs do not have non-specific, or non-desirable, binding properties, as indicated by binding to non-target moieties. This is achieved by a selection/counter-selection paradigm in which mAbs are exposed to the target Ag and one or more non-target moieties and the microfluidics device is used to identify and collect droplets containing mAbs that bind to the target Ag to a significantly greater extent than they bind to the non-target moieties. In one embodiment of this approach, the target Ag is attached to a bead or other particle that acts as a solid support. Particles carrying target moieties, optionally referred to in some cases as “Ag-coated beads” can be used for selection, i.e., to identify those candidate binding moieties (e.g., candidate mAbs) that bind to the target, and second type or secondary beads can be used for counter-selection, i.e., to identify those candidate binding moieties (e.g., again, mAb) that do or do not bind preferentially to the target. That is, binding specificity can be determined by examining binding differential in a droplet between Ag-coated beads and non-target coated secondary beads. An example of this dual bead approach is shown in FIG. 9, wherein hybridoma cells are incubated in a droplet containing two different bead-types, one coated with the target Ag, and a second bead-type that is coated with a non-target moiety, i.e., a moiety to which it is not desirable for the selected mAb to bind.

The target and non-target moieties are optionally associated with beads by chemical conjugation or, alternatively, by a high affinity, non-covalent interaction such as that between biotin and streptavidin, as a non-limiting example. Such methods for attachment of Ag to a solid support are well known in the art; one non-limiting example is the direct chemical coupling of Ag to carboxylated 5 micron polystyrene divinyl-benzene copolymer beads through lysine residues within the Ag. A second non-limiting example is generation of a fusion protein between the Ag and biotin or a biotin derivative and then attachment the said fusion protein to streptavidin beads.

In FIG. 9, hybridoma cells secreting mouse mAbs into the droplet medium are incubated in droplets with two types of beads: one coated with a target Ag and a secondary bead, optionally coated with a non-target moiety. The droplets are then merged with a droplet containing a fluorescently-labeled secondary Ab directed against mouse Abs. The droplet is then passaged through a narrowing in the flow channel such that the bead(s) flow past a detector in single-file. The location of the secondary Ab is then measured and droplets having both fluorescently-labeled Ag beads and unlabeled secondary beads are sorted and selected. In alternative embodiments, the secondary bead can be coated with multiple different non-target Ags. In yet other embodiments, multiple different secondary beads are used, each such secondary bead carrying one or more different non-target moieties, such that the Ag-binding bead can be distinguished from all secondary beads. In yet another embodiment, ‘naked” beads are used to reveal mAbs that are binding to the bead moiety of the Ag-bonding bead, as opposed to the target Ag per se.

The first and second bead types, e.g., antigen-coated beads and secondary beads, can variably differ in any way that allows them to be differentiated by a suitable readout, including, in non-limiting examples, by size, shape, color and/or fluorescence signal. In one non-limiting example the beads are differentiated by a difference in size. In another non-limiting example, Ag-coated beads and secondary beads are differentiated based upon the presence or absence of one or more molecules that allow the beads to be distinguished colorimetrically or fluorimetrically. In one such non-limiting example, the target Ag is detectably labeled with a chromogenic or fluorescent molecule, such that only Ag-coated beads are labeled. In a further non-limiting example, the non-target moiety is detectably labeled chromogenic or fluorescent molecule, such that only secondary beads are labeled.

In yet other non-limiting examples of the methods, systems and devices disclosed here, the beads contain differentiating label, such that the bead component of the Ag-coated bead is labeled whereas the bead component of the secondary bead is not; or alternatively such that the bead component of the secondary bead is labeled whereas the bead component of the Ag-coated bead is not; or alternatively such that the bead component of the Ag-coated bead and the secondary bead are both labeled, but can be distinguished by a detection process. In a preferred embodiment of this non-limiting example, squaric dyes are used, as in the case of beads made by Luminex Corporation (Austin, Tex.). Accordingly, as illustrative examples, Ag-coated beads contain such squaric dyes whereas secondary beads do not; or secondary beads contain such squaric dyes whereas Ag-coated beads do not; or both Ag-coated beads and secondary beads contain squaric dyes such that they can nevertheless be differentiated on the basis of their fluorescence signal.

There are a number of methods described in the art for detecting the presence of, and monitoring the quantity of, Abs bound to Ag. The applicability of such methods to various embodiments of the present disclosure will be apparent to those of ordinary skill in the art given the benefit of this disclosure. One of these methods is used to measure the amount of Ab bound per Ag-coated bead vs the secondary bead. As one non-limiting example, a labeled secondary Ab is used that binds to the Abs being evaluated for their ability to bind to target Ag. In the illustrated embodiment of FIG. 9, the secondary Ab binds to mouse Ab (e.g., a fluorescein-labeled anti-mouse Ab, although other labels are known in the art). In instances in which other Ab species are being evaluated, as, for example, human Ab, or, in one embodiment, human scFv fragments, the secondary Ab used is one that binds to these Abs. In the event that secondary Ab and bead are both detected via the same type of signal, for example fluorescence, it is a requirement that these signals be distinguishably detectable. In another non-limiting example, the Ab is produced as part of a fusion protein also containing GFP, such that the fusion protein can be located and quantitated by the green fluorescence emitted by GFP. This latter example is particularly suitable, for example, in instances in which hybridomas are not used as the source of Ab.

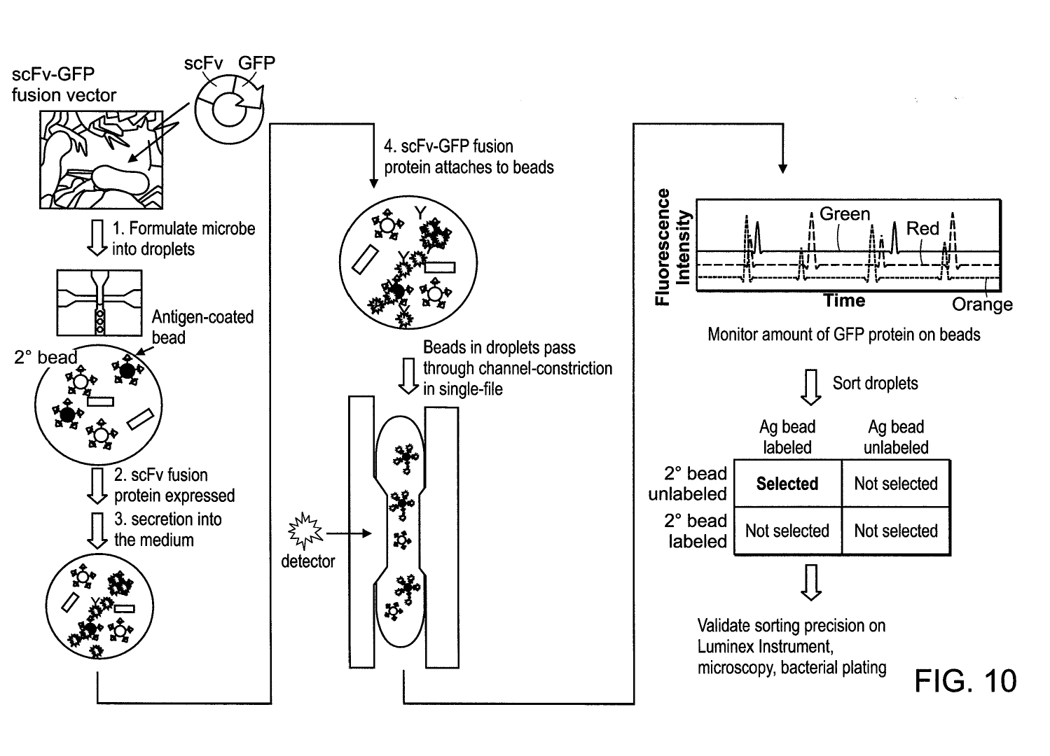

FIG. 10 illustrates a microfluidics-based approach for the isolation of Ag-specific Ab using a dual bead specificity assay and secreted scFv-GFP fusion protein. In micro-organisms able to secrete protein directly into the culture medium (for example, Bacillus subtilis and other Gram-positive organisms, and yeast and other eukaryotes) a leader peptide is genetically fused to the 5′ end of a scFv-GFP fusion gene. This leader peptide is adequate to functionalize the transport of the protein into a) the periplasmic space (i.e., the layer external to the inner membrane) in Gram-positive micro-organisms, and b) the endoplasmic reticulum (in eukaryotes, including especially Pichia and S. cerevisiae). The micro-organisms are encapsulated into droplets. The bacterial and eukaryote hosts continue to express the fusion protein within the droplets. In the embodiment shown, at least two differentiatable 5-micron Luminex bead-types are each separately coated with different Ags (in the figure, they are labeled as “antigen-coated bead” and “2° bead”, wherein the Ag-coated bead is the selection bead to which target Ag has been attached, and the secondary bead is the counter-selection bead, to which a non-target moiety has been attached). The two bead-types are co-encapsulated with host cells expressing the fusion protein within droplets approximately 30 μm in diameter and an emulsion of at least 108 droplets collected. The droplet emulsion is incubated to allow sufficient protein production and then re-injected into the microfluidics device. The beads are passed through a constriction in the channel such that beads passage through in single-file. The bead-type and amount of fluorescence associated with each bead are determined. Droplets containing beads in which the amount of GFP fluorescence per Ag-coated bead exceeds the amount of GFP fluorescence per secondary bead is above a given threshold are collected. In a further embodiment of this invention, collected beads are optionally re-analyzed on a Luminex instrument, and by plating and analyzing colonies in the sorted emulsion by DNA sequencing, Western and ELISA analyses. If the Ag-containing bead is labeled (with GFP) and the secondary bead is not labeled (or if difference in the amount of labeling exceeds a given threshold) then binding of mAb in the droplet is considered to be specific to the target Ag. If both bead-types are labeled and the difference in labeling does not exceed the set threshold, then mAb binding is considered to be non-specific. If only the secondary bead is labeled in a droplet, then the mAb being made in that droplet is either non-specific or specific for the Ag coating the secondary bead. If neither bead-type is labeled, then either the droplet does not contain an mAb-producing cell, or the Ab that is produced does not recognize Ag on either bead-type.

After droplets are evaluated for the relative binding of mAb to Ag-coated beads vs the secondary beads, they can be gated such that only droplets in which the amount of mAb bound per Ag-coated bead exceeds the amount of mAb bound per secondary bead by a pre- or experimentally-determined threshold amount are sorted for further analysis. Other droplets are rejected from further consideration. In one such embodiment droplets are selected for further analysis if the amount of mAb binding per Ag-coated bead is at least two-fold greater than the amount of mAb binding per secondary bead, although droplets in which there is a greater ratio of mAb binding per Ag-coated bead vs mAb binding per secondary bead are preferentially selected for further analysis.

Although hybridomas are Ab-producing cells, they possess limitations with regard to use in microfluidics. Other cells types have the advantages over hybridomas that they survive longer in droplets, have shorter doubling times and are less expensive to grow and maintain. As a non-limiting example, a eukaryotic cell line can be used other than a hybridoma. Non-limiting examples of such eukaryotic cell lines are insect and mammalian cell lines, both of which are known to secrete certain proteins directly into the milieu and in which leader peptide sequences are known in the art to promote the secretion of such proteins. As further non-limiting examples, a display vector, such as baculovirus (in the case of insect cells) or adenovirus (in the case of mammalian cells), and the like, are used to display Abs in the milieu. In a non-limiting example of an embodiment in which cells other than hybridoma cells are used, a library of Abs is generated in a cell. In a further non-limiting example, a library of Abs containing a synthetic human framework is generated in a cell. In a third non-limiting example, the Abs in the library are scFv. In a fourth non-limiting example, the Ab library contains between 10⁶ and 10⁹ members. In a preferred embodiment, the Ab library contains between 5×10⁷ and 5×10⁸ members. Also in a preferred embodiment, the micro-organism containing the Ab library to be screened secretes proteins freely into the milieu rather than into a periplasmic space (i.e., the space between the cell wall and the outer membrane) and the Abs generated are secreted by the micro-organism. As a non-limiting example, the micro-organism is Bacillus subtilis, which is known to secrete proteins directly into the milieu. As another non-limiting example, the micro-organism is a fungus, such as a member of the genus Pichia, or Saccharomyces, both of which are known to secrete proteins directly into the milieu.

Michael Weiner, a named inventor of this invention, has described construction of a naïve scFv library (patent application 20060160178). In one embodiment, the same library-construction method is used in the present invention. Also in the present invention, in one embodiment a framework is used that enables efficient expression in Bacillus subtilis and results in the secretion of functionally active scFv into the milieu. In one embodiment, the library is cloned into a Bacillus subtilis-E. coli bifunctional plasmid encoding chloramphenical resistance (which is effective in both organisms), pC194 derivative. The Ab gene is, in one embodiment, flanked by a leader sequence and a reporter gene. In one such embodiment, Ab genes are cloned 3′ (downstream) of an amylase leader peptide and 5′ to GFP. In such embodiment, the final fusion protein construct is in the form: (amino terminus) leader peptide-Ab-GFP protein (carboxyl terminus). In a second embodiment, the reporter gene is an enzyme such as β-Galactosidase or alkaline phosphatase (or any of several other enzymes known in the art as reporters), in which case the final fusion protein construct is in the form: (amino terminus) leader peptide-Ab-β-Galactosidase or (amino terminus) leader peptide-Ab-alkaline phosphatase, respectively. In a preferred embodiment, the hybrid genes in Bacillus are placed under control of a late spore promoter such as spoV. All clones are transformed into, and expressed in, a strain of Bacillus subtilis, protease-minus strain YB886 being one non-limiting example. Unlike E. coli, Bacillus is naturally transformable. Thus the Ab genes can be first cloned into E. coli and plasmid DNA clones used to transform the Bacillus, preferably to more than 10⁸ individual isolates. In order to prevent the biased outgrowth of a limited number of clones, all cell growth can be done on solid medium.

In further embodiments, as illustrated in FIG. 10, protein or peptide Ags, either containing PTMs or unmodified, are prepared (variously by, for example, purification from mammalian cells or synthesis de novo). The embodiments illustrated in FIG. 10 use a microfluidics-based approach for the isolation of Ag-specific Ab using a dual bead specificity assay and secreted (into the droplet) scFv-GFP fusion protein. mAb are screened for specificity against the desired Ag by the use of Ag-coated beads and secondary beads as described above. In this particular embodiment, Ag-coated beads have the target Ag (either an unmodified or a post-translationally modified protein or protein epitope) attached whereas secondary beads have one or more of the non-target versions of the protein or epitope attached. In one non-limiting example, if it is desired to obtain mAbs that differentially bind to the unmodified version of a protein or epitope but not to a version modified by PTM, Ag-coated beads have the unmodified version attached and secondary beads have one or more versions attached. In a second non-limiting example, if it is desired to obtain mAbs that differentially bind to a version of a protein or epitope modified by PTM but not to an unmodified version, Ag-coated beads have the unmodified version attached and secondary beads have the unmodified version attached. In a third non-limiting example, if it is desired to obtain mAbs that differentially bind to a particular modified version of a protein or epitope but not to a differently modified version, Ag-coated beads have attached the modified version of the protein or epitope that is being targeted, whereas secondary beads have the differently modified version(s), and optionally, the unmodified version, of the protein or epitope attached. As described above for FIG. 10, Ab-producing cells secrete mAb in a droplet containing both Ag-coated beads and secondary beads and after suitable incubation, droplets are monitored for the association of mAb with the Ag-coated beads and/or secondary beads. Following such analysis, mAb associated only, or predominantly, with Ag-coated beads (as opposed to secondary beads) are selected. Once again, although in the current embodiment bacterial cells producing Ab are the preferred embodiment, in alternative embodiments, lower or higher eukaryotic cells, including hybridoma cells, are used to produce Ab, which are then selected for specificity as well as affinity for target by the dual bead approach herein described.

The mAb prepared in this non-limiting example as scFv fused with GFP can be produced by Bacillus and are screened for specificity against the desired Ag by the use of Ag-coated beads and secondary beads. Ab-producing cells secrete mAb in a droplet containing both Ag-coated beads and secondary beads. After suitable incubation, droplets are monitored for the association of the antibody with Ag-coated beads and secondary beads. Following such analysis, Ab associated only, or predominantly, with Ag-coated (as opposed to secondary beads) are selected. In micro-organisms able to secrete protein directly into the culture medium (for example, Bacillus subtilis and other Gram-positive organisms, and yeast and other eukaryotes) a leader peptide is genetically fused to the 5′ end of a scFv-GFP fusion gene. This leader peptide is adequate to functionalize the transport of the protein into a) the periplasmic space (i.e., the layer external to the inner membrane) in Gram-positive micro-organisms, and b) the endoplasmic reticulum (in eukaryotes, including especially Pichia and S. cerevisiae). The micro-organisms are encapsulated into droplets. The bacterial and eukaryote hosts continue to express the fusion protein within the droplets. In the embodiment shown, at least two differentiatable 5-micron Luminex bead-types are each separately coated with different Ags (in the figure, they are labeled as “antigen-coated bead” and “2° bead”, wherein the antigen-coated bead is the selection bead to which target Ag has been attached, and the secondary bead is the counter-selection bead, to which a non-target moiety has been attached). The two bead-types are co-encapsulated with host cells expressing the fusion protein within droplets approximately 30 μm in diameter and an emulsion of at least 10⁸ droplets collected. The droplet emulsion is incubated to allow sufficient protein production and then re-injected into the microfluidics device. The beads are passed through a constriction in the channel such that beads passage through in single-file. The bead-type and amount of fluorescence associated with each bead are determined. Droplets containing beads in which the amount of GFP fluorescence per Ag-coated bead exceeds the amount of GFP fluorescence per secondary bead is above a given threshold are collected. In a further embodiment of this invention, collected beads are optionally re-analyzed on a Luminex instrument, and by plating and analyzing colonies in the sorted emulsion by DNA sequencing, Western and ELISA analyses. If the Ag-containing bead is labeled (with GFP) and the secondary bead is not labeled (or if difference in the amount of labeling exceeds a given threshold) then binding of mAb in the droplet is considered to be specific to the target Ag. If both bead-types are labeled and the difference in labeling does not exceed the set threshold, then mAb binding is considered to be non-specific. If only the secondary bead is labeled in a droplet, then the mAb being made in that droplet is either non-specific or specific for the moiety coating the secondary bead. If neither bead-type is labeled, then either the droplet does not contain an mAb-producing cell, or the Ab that is produced does not recognize Ag on either bead-type.

One advantage that a microfluidics-based Ab selection approach has over the yeast two-hybrid assay is that yeast cells do not post-translationally modify proteins (phosphorylation, acetylation, glycosylation, etc.) in the same way that higher eukaryotic cells do. Microfluidic screening can be used to select suitable mAbs directed against post-translationally modified proteins, or against non-modified versions of proteins that can be post-translationally modified. Such proteins (or peptidic epitopes thereof) can be obtained in various ways well known in the art, including, as non-limiting examples, purification from their native source or synthesis de novo followed by modification, when appropriate, either in vivo or in vitro.

In a number of the foregoing embodiments, the mAb are produced in micro-organisms, such as Bacillus subtilis, that are able to secrete protein directly into the culture medium. Other embodiments of the current invention make use of bacteriophage to transport mAb outside the micro-organism and into droplets where they can come into contact with Ag. In a preferred embodiment, the vector used is bacteriophage M13. In E. coli, a leader peptide placed on a protein exports that protein into the periplasmic space (i.e., the layer between the inner and outer membrane), but generally not into the medium. Bacteriophage M13 is able to extrude itself through the E. coli outer membrane and for that reason it is a reasonable agent for use in this invention. M13 is composed of circular single stranded DNA (6407 nucleotides long) encapsulated in approximately 2700 copies of the major coat protein p8, and capped with 5 copies of two different minor coat proteins (p9, p6, p3) on the ends. The minor coat protein P3 attaches to the receptor at the tip of the F-pilus of the host E. coli. Infection with filamentous phages such as M13 is not lytic. The phage M13 is a virus that, upon, infection gives rise to turbid plaques in E. coli. The size of the M13 phage particle is determined by the number of bases the phage packages. Once the phage DNA has been fully coated with p8, the secretion terminates by addition of the p3/p6 cap, and the new phage detaches from the bacterial surface. New M13 phage particles are secreted within 10 minutes from a newly infected host and can arise at a rate of 1000 plaque forming units (pfu) per cell within the first hour of infection. The bacterial host can continue to grow and divide, allowing this process to continue indefinitely.

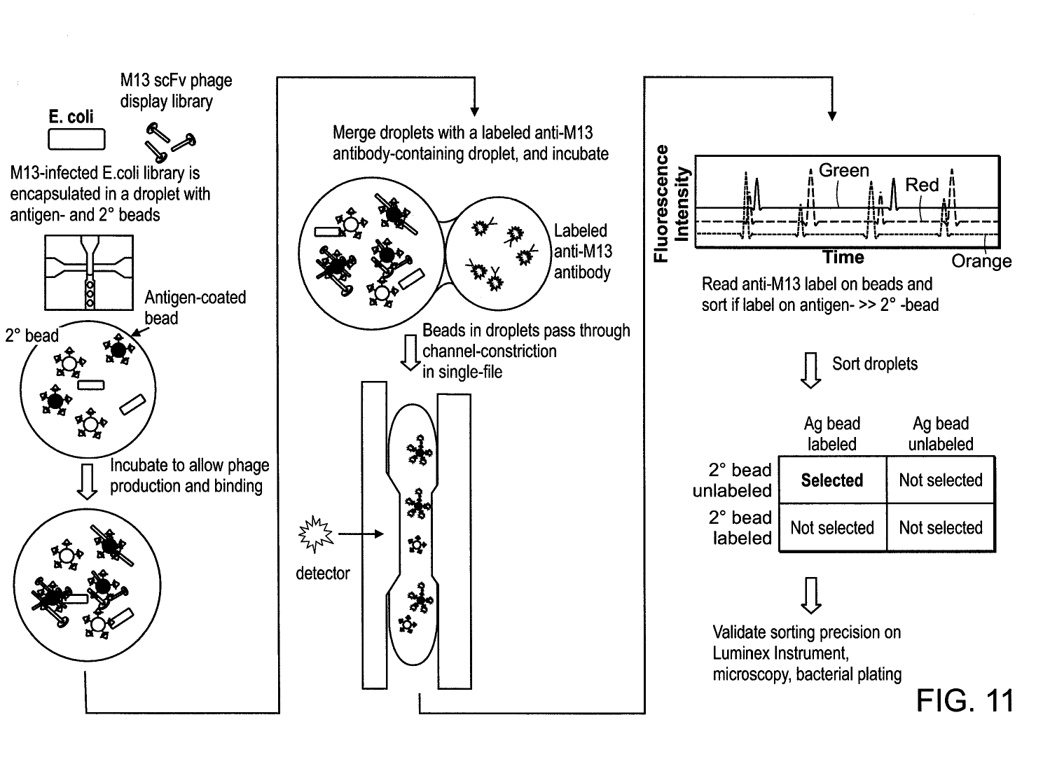

A non-limiting example of the isolation of Ag-specific mAb using dual bead specificity assay and M13-based system, specifically, the use of bacteriophage M13 to identify selective mAb is illustrated in FIG. 11. In the example shown there, the M13 gp3 protein is fused to an scFv. As further illustrated, a second droplet is used to introduce a fluorescently-labeled anti-M13 Ab. In other non-limiting examples, it is preferable to fuse a chimeric construct of GFP-scFv to the gp3 protein and thereby obviate the need for the second droplet combining.

Two different 5-micron Luminex bead-types are each separately coated with different Ags (labeled as “antigen-coated bead” and “2° bead”), wherein the Ag-coated bead is the selection bead and the secondary bead is the counter-selection bead. The two bead types are co-encapsulated with M13-infected E. coli within 30 μm (or slightly larger) droplets and an emulsion of at least 10⁸ droplets is collected. The droplet emulsion is incubated to allow sufficient phage production and then re-injected and merged with droplets containing fluorescently-labeled anti-M13 Ab and incubated for a sufficient additional period of time to generate mAb—Ag complexes. The beads are passed through a constriction in the channel such that beads passage through in single-file. The bead-type and amount of fluorescence covering the bead are detected. Droplets containing beads with a concentration of fluorescence on Ag-coated beads that exceeds the concentration of fluorescence on the secondary beads above a given threshold are collected. The microfluidics results are optionally re-analyzed on a Luminex instrument, and/or by plating and analyzing phage in the sorted emulsion by DNA sequencing, Western and/or ELISA analysis.

Many instances can be contemplated in which it is desirable to identify Abs that bind specifically to non-protein targets. Examples are known in the art in which Abs have been isolated that bind to nucleic acids, carbohydrate-containing chains and the like. It is often difficult to obtain Ab that bind to such non-protein targets with high degree of affinity and specificity. In many cases, non-protein target Ags, particularly small molecules are used as haptens, i.e., they are bound to carrier proteins in an effort to generate and identify suitable Abs. In the present invention, there is no need to immunize an animal to obtain Abs; an already-formed Ab library is screened vs the target Ag for the presence of a suitable binder. Thus carrier protein is not required in the present invention in the process of selecting mAb that bind to non-protein Ags. In one embodiment of the present invention, the Ag is conjugated to a particle, a bead being a non-limiting example, by any of several means described in the art, the method chosen depending upon the chemical nature of the Ag. mAb selection is then carried out by any of the appropriate procedures described herein, including the used of naked beads or beads containing non-target moieties to increase the likelihood that selected mAbs are specific for their target Ags.

From the foregoing disclosure and detailed description of certain exemplary embodiments, it is also apparent that various modifications, additions and other alternative embodiments are possible without departing from the true scope and spirit of the present invention. Thus, in the examples above, representations of several of many variations of the methods and systems of the invention are shown and described, including the use of phage to display the scFv, and direct secretion of a fusion-scFv directly into the milieu. These are meant merely to illustrate the basic concepts of the invention disclosed herein. Many other embodiments or versions of the disclosed invention will be apparent to those skilled in the art given the benefit of this disclosure, including, but not limited to, methods, apparatus and systems of the invention employing baculoviral display, ribosome binding display, flagella display, yeast surface display, phage lambda and/or T7 display. Similarly, variations in the apparatus, target Ag or species, etc. will be apparent to those skilled in the art given the benefit of this disclosure.

It should be understood that all examples given in this description of various embodiments of the disclosed methods, systems and devices on non-limiting examples. Variations and alternatives involving different or additional steps, features or the like should be understood to be within the scope of the disclosed inventive subject matter. It should be understood that the present disclosure is intended to include all feasible and operative combinations and permutations of the numerous alternative features, method steps and the like of the various embodiments discussed, whether or not such particular combination or permutation was expressly mentioned.

1. An interaction screening method for identifying one or more candidate binding moieties based on binding to one or more known other moieties comprising:
a. providing droplets of liquid medium, at least a majority of which contain at least one recombinant binding moiety as a candidate binding moiety, a first known moiety, and a second known moiety, the candidate binding moiety of some or all droplets being different from the candidate binding moiety of some or all other droplets; and
b. determining for one or more of the droplets whether the candidate binding moiety is bound to the first known moiety and whether the candidate binding moiety is bound to the second known moiety.

2. The interaction screening method of claim 1 for identifying one or more candidate binding moieties based on binding to one or more known other moieties, further comprising segregating from others of the droplets at least one droplet in which the candidate binding moiety is bound to the first known moiety.

3. The interaction screening method of claim 2 for identifying one or more candidate binding moieties based on binding to one or more known other moieties, wherein at least one of the first known moiety and the second known moiety is a known antigen and the candidate binding moieties are antibodies.

4. (canceled)

5. The interaction screening method of claim 2 for identifying one or more candidate binding moieties based on binding to one or more known other moieties, wherein the first known moiety and the second known moiety are carried in the droplet by support particles.

6. The interaction screening method of claim 1 for identifying one or more candidate binding moieties based on binding to one or more known other moieties, further comprising:
identifying a cell that produces a binding moiety contained in one or more of the droplets identified in step (b); and
producing the binding moiety comprising multiplying the cell.

7. (canceled)

8. (canceled)

9. An interaction screening method for identifying one or more candidate binding moieties based on binding to a target binding moiety, comprising:
a. providing droplets of liquid medium which contain at least one recombinant binding moiety as a candidate binding moiety and at least one target binding moiety, the candidate binding moiety of some or all of the droplets being different from the candidate binding moiety of some or all others of the droplets; and
b. determining for one or more of the droplets whether the candidate binding moiety is bound to the target binding moiety.

10. (canceled)

11. The interaction screening method of claim 9 for identifying one or more candidate binding moieties based on binding to a target moiety, wherein the droplets are provided in step (a) by steps comprising at least forming an emulsion in oil of droplets of an aqueous dispersion of recombinant binding moieties.

12. (canceled)

13. (canceled)

14. The interaction screening method of claim 9 for identifying one or more candidate binding moieties based on binding to a target moiety, wherein the recombinant binding moiety of each of at least a portion of the droplets is provided in the droplet in step (a) by viral, phage or yeast display or bacterial, archea or eukaryote secretion.

15. The interaction screening method of claim 9 for identifying one or more candidate binding moieties based on binding to a target moiety, wherein the recombinant binding moieties are selected from the group consisting of antibodies, proteins, and nucleic acids.

16. The interaction screening method of claim 9 for identifying one or more candidate binding moieties based on binding to a target moiety, wherein at least some of the target moieties are carried on support particles in the droplets.

17.-20. (canceled)

21. A screening method for identifying one or more candidate binding moieties based on binding to a target moiety, comprising:
a. providing a collection of microbes in a culture medium suitable for incubation of the microbes to express a library of candidate binding moieties, different ones of which microbes express at least one candidate binding moiety different from candidate binding moieties provided by some or all others of the microbes;
b. in any order—
i. placing at least a portion of the microbes into a stream of droplets of the culture medium in a microfluidic channel,
ii. providing in each of at least some of the droplets one or more first support particles carrying a target moiety, and
iii. incubating at least a portion of the droplets; and
c. then identifying one or more of the droplets in which the corresponding candidate binding moiety is bound to the target moiety, comprising:
i. exposing at least a portion of the droplets to a detector, and
ii. operating the detector to detect droplets, if any, in which the candidate binding moiety is bound sufficiently to the one or more first solid support particles in the droplet.

22.-55. (canceled)

56. The interaction screening method in accordance with claim 21 for identifying one or more candidate binding moieties based on binding to a target moiety, wherein step (c) comprises transporting at least a portion of the droplets in a microfluidic channel past a detector.

57. The interaction screening method in accordance with claim 21 for identifying one or more candidate binding moieties based on binding to a target moiety, wherein step (c) comprises collecting a one-thick film of droplets onto a surface, identifying droplets in the film, if any, in which the candidate binding moiety is bound to the one or more first solid support particles in the droplet.

58.-69. (canceled)

70. The interaction screening method in accordance with claim 21 for identifying one or more candidate binding moieties based on binding to a target moiety, wherein the droplets further contain one or more known moieties additional to the target moiety, and step (c) further comprises identifying one or more of the droplets in which the candidate binding moiety is bound to at least one of the additional known moiety.

71.-81. (canceled)

82. The interaction screening method in accordance with claim 21 for identifying one or more candidate binding moieties based on binding to a target moiety, further comprising:
d. recovering at least one microbe from at least one droplet identified in step (c);
e. producing the candidate binding moiety of the droplet recovered in step (d), comprising cultivating the microbe recovered in step (d) under conditions in which expression of the gene encoding the candidate binding moiety occurs.
